(19) United States
(12) Patent Application Publication
(10) Pub. No.: US 2014/0011246 A1
(43) Pub. Date: Jan. 9, 2014

(54) Methods for Harvesting and Processing Biomass

(71) Applicants: Ronald Sims, Logan, UT (US); Charles Miller, North Logan, UT (US); Joshua T. Ellis, Logan, UT (US); Ashik Sathish, North Logan, UT (US); Renil Anthony, Salt Lake City, UT (US); Asif Rahman, Logan, UT (US)

(72) Inventors: Ronald Sims, Logan, UT (US); Charles Miller, North Logan, UT (US); Joshua T. Ellis, Logan, UT (US); Ashik Sathish, North Logan, UT (US); Renil Anthony, Salt Lake City, UT (US); Asif Rahman, Logan, UT (US)

(73) Assignee: Utah State University, North Logan, UT (US)

(21) Appl. No.: 13/915,612

(22) Filed: Jun. 11, 2013

Related U.S. Application Data
(60) Provisional application No. 61/657,972, filed on Jun. 11, 2012.

(51) Int. Cl. C12P 7/64; C12P 5/02; C12P 7/28; C12P 7/16; C12P 7/62; C12P 7/06

(52) U.S. Cl. CPC C12P 7/649; C12P 7/625; C12P 7/065; C12P 7/28; C12P 7/16; C12P 5/023
USPC 435/134; 435/289.1; 435/257.1; 435/135; 435/161; 435/150; 435/160; 435/167

(57) ABSTRACT

A system and method for harvesting and processing algae, the system and method including harvesting algae by mechanical or chemical system and processing the harvested algae to produce at least one of biodiesel, biosolvents, bioplastics, biogas, or fertilizer.

### CROSS REFERENCE TO RELATED APPLICATIONS

This application claims priority to U.S. Provisional Patent Application No. 61/657,972, filed Jun. 11, 2012, the entirety of which is hereby incorporated by reference. This application hereby incorporates by reference the following related U.S. patent application Ser. Nos.: 12/907,572, filed Oct. 19, 2010; 13/660,161, filed Oct. 25, 2012; 13/663,002, filed Oct. 29, 2012; 13/663,315, filed Oct. 29, 2012; and 13/914,461, filed Jun. 10, 2013.

### GOVERNMENT SPONSORED RESEARCH

The inventions described herein were made at least in part with government support under contract DE-EE0003114 awarded by the United States Department of Energy. The government has certain rights in the inventions.

### TECHNICAL FIELD

The present disclosure relates to methods of harvesting and processing biomass, more particularly, it relates to methods of harvesting and processing algae into bioproducts.

### BACKGROUND

The production of bioproducts from various biological feedstocks has been explored in an effort to produce high-value products from renewable and/or inexpensive feedstocks. However, improved methods, systems, and apparatuses are needed for commercial viability and/or feasibility to be established.

In particular, algae have been identified as a potential biological feedstock in numerous applications. Various methods and/or apparatuses of harvesting and processing algae have been described. However, additional and efficient methods for harvesting and processing algae are needed for algae to serve as a large-scale biological feedstock and biomass source.

### SUMMARY

The present disclosure in aspects and embodiments addresses these various needs and problems by providing systems, methods, and apparatuses for harvesting and processing algae and other bio-feed stocks. These systems, methods, and apparatuses may be integrated into biomass harvesting and processing systems where feedstocks are harvested, separated into various phases, and processed into various high-value bioproducts. The systems may be interdependent and may be adjusted as the bioproduct market fluctuates to provide for a total system that is flexible enough to provide for an economically viability and commercially feasible system of processing biomass, particularly algae.

The methods, systems, and apparatuses provide a system and method for harvesting and processing algae, the system and method including harvesting algae by mechanical or chemical system and processing the harvested algae to produce at least one of biodiesel, biosolvents, bioplastics, biogas, or fertilizer.

### BRIEF DESCRIPTION OF THE DRAWINGS

FIG. 1 illustrates a flow diagram of an exemplary harvesting and processing system.

FIG. 2 shows a rotating bioreactor partially submerged in liquid media with rope type substratum wound onto cylinder for biofilm growth.

FIG. 3 shows a harvesting apparatus in conjunction with a rotating bioreactor.

FIG. 4 shows a multiple cylinder setup.

FIG. 5 shows a rotating reactor within a flotation frame.

FIG. 6 shows a high rate algal pond with associated rotating bioreactors.

FIG. 7 shows the photosynthetically active radiation cycle of bench scale reactors when operated at 4.8 rpm.

FIG. 8 shows the growth curves of a suspended culture, initial biofilm culture, and secondary biofilm culture.

FIG. 9 shows soluble P removal rates and soluble P concentrations of the suspended and biofilm reactors.

FIG. 10 shows soluble N removal rates and soluble N concentrations of the suspended and biofilm reactors.

FIG. 11 pH based zeta potential comparison of cationic corn, cationic potato starch and alum

FIG. 12 H-NMR of unmodified corn starch

FIG. 13 H-NMR of corn cationic starch graftd polymer

FIG. 14 Comparison of TSS removal from Logan lagoon waste water using cationic corn and potato starch, and alum

FIG. 15 Comparison of total phosphorus removal from Logan lagoon wastewater using cationic corn and potato starch, and alum

FIG. 16 illustrates an exemplary method of producing biodiesel.

FIG. 17 illustrates the precipitation of algal pigments that occurs using an exemplary method.

FIG. 18 illustrates a flow diagram according to an exemplary embodiment.

FIG. 19 illustrates production yields according to an exemplary embodiment.

FIG. 20 illustrates production yields according to an exemplary embodiment.

FIG. 21 illustrates production yields according to an exemplary embodiment.

FIG. 22 illustrates production yields according to an exemplary embodiment.

FIG. 23 illustrates production yields according to an exemplary embodiment.

FIG. 24 illustrates production yields according to an exemplary embodiment.

FIG. 25 illustrates production yields according to an exemplary embodiment.

FIG. 26 illustrates a CFU/mL for various exemplary samples.

FIG. 27 is an NMR for a product produced according to an exemplary plastic production method.

FIG. 28 is an NMR for a product produced according to an exemplary plastic production method.

FIG. 29 is an NMR for a product produced according to an exemplary plastic production method.

FIG. 30 is an NMR for a product produced according to an exemplary plastic production method.

FIG. 31 is an NMR for a control product.

FIG. 32 is an NMR for a product produced according to an exemplary plastic production method.

FIG. 33 is an NMR for a product produced according to an exemplary plastic production method.

FIG. 34 is an NMR for a product produced according to an exemplary plastic production method.

FIG. 35 illustrates OD600 v. time for different concentrations of glycerol in M9 media.

FIG. 36 is an NMR for exemplary PHB secreting strains.

### DETAILED DESCRIPTION

The present disclosure covers methods, compositions, reagents, and kits for systems of biomass harvesting and processing. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

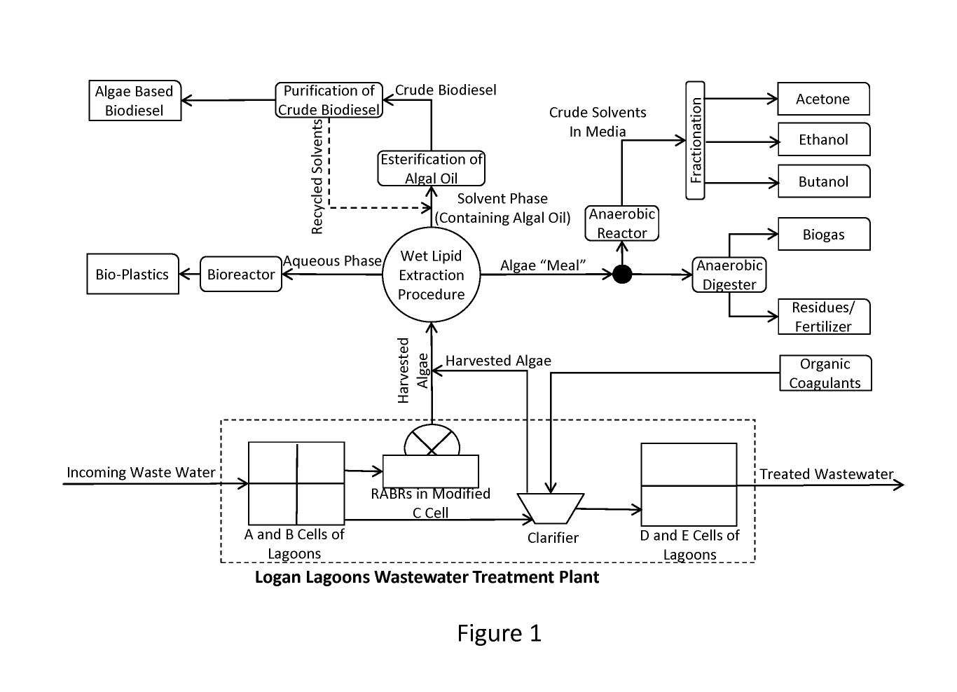

An exemplary system of harvesting and processing biomass is illustrated in FIG. 1. The system may include harvesting biomass mechanically, harvesting biomass chemically, extracting lipids, solids, and glycerol from harvested biomass, processing extracted lipids to produce biodiesel, processing solids to produce solvents, and processing glycerol to produce bioplastics.

### I. Harvesting Biomass

Any suitable harvesting method or methods may be used alone or in combination to harvest biomass. Exemplary methods, apparatuses, and compositions that may be used alone or in combination for harvesting algal biomass include, mechanical and chemical harvesting techniques.

A. Rotating Bioreactor

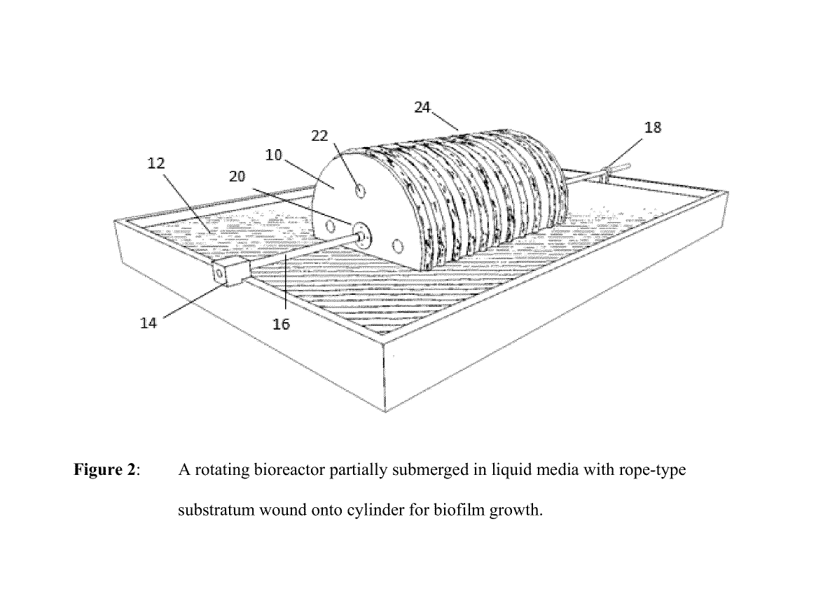

A rotating bioreactor apparatus as described in U.S. patent application Ser. No. 13/040,364, filed Mar. 4, 2011, which claims priority to U.S. Patent Application No. 61/310,360, filed Mar. 4, 2010 (the entirety of which is herein incorporated by reference) may be used to harvest biomass. In FIG. 2 there is shown a body 10 partially submerged in a liquid medium 12. In this embodiment the body is in the form of a right circular cylinder. Additional body formats may be utilized including, but not limited to, elliptic cylinder, parabolic cylinder, hyperbolic cylinder, generalized cylinder or oblique cylinder or any form with a rotational axis suitable for this purpose.

One skilled in the relevant art will recognize that different formulations of liquid medium 12 will be used to produce different types of biomass. The liquid medium 12 may be a complex, defined, or selective growth medium. More specifically, the liquid medium 12 may be a complex medium including, but not limited to complex dextrose based media, sea water media, domestic wastewater, municipal wastewater, industrial wastewater, surface runoff wastewater, soil extract media, or any natural water containing detectable amounts of phosphorus or nitrogen; or a defined medium, including, but not limited to Bristol's medium, Bolds Basal medium, Walne medium, Guillard's f medium, Blue-Green medium, D medium, DYIY medium, Jaworski's medium, K medium, MBL medium, Jorgensen's medium, and MLA medium; or a selective medium including, but not limited to minimal media based on specific nutrient auxotrophy, and selective media that incorporates antibiotics. Depending on the chosen liquid medium 12 and seed culture, the resulting biofilm may be a mixed or pure culture and may be comprised of microalgae, cyanobacteria, nitrifying bacteria, heterotrophic bacteria, microscopic fungi, or any combination thereof.

Still referring to FIG. 2, a rotation device 14 transmits rotational power to a drive shaft 16 that runs through the center of the cylinder 10 and is supported by a bearing 18 opposite the rotation device 14. Where the drive shaft 16 enters and exits the cylinder 10, a base plate 20 is used to connect the drive shaft 16 and the cylinder 10. Holes 22 are made in the ends of the cylinder 10 to allow liquid media 12 to enter the cylinder 10. A substratum 24 is placed around the cylinder 10 for biofilm growth.

In more detail, still referring to FIG. 2, the rotation device 14 transmits rotational power to the drive shaft 16, causing the cylinder 10 to rotate with the drive shaft 16. As the cylinder 10 rotates, the biofilm substratum 24 placed on the surface of the cylinder 10 is alternately exposed to the liquid media 12 and the air.

In further detail, still referring to FIG. 2, the biofilm substratum 24 may be in the form of a rope, cable or belt or the like such that it can be wound around the outer circumference of the cylinder 10. The substratum 24 may be selected from a group comprising cotton, jute, hemp, manila, silk, linen, sisal, silica, acrylic, polyester, nylon, polypropylene, polyethylene, polytetrafluoroethylene, polymethylmethacrylate, polystyrene, polyvinyl chloride, or any other non-rigid material capable of supporting biofilm growth. One end of the substratum 24 is attached to one end of the surface of the cylinder 10 and wound around until the surface of the cylinder 10 may be sufficiently covered with the substratum 24. The free end of the substratum 24 may then be attached to the surface of the cylinder 10 to keep the substratum 24 from unwinding during rotation of the cylinder 10.

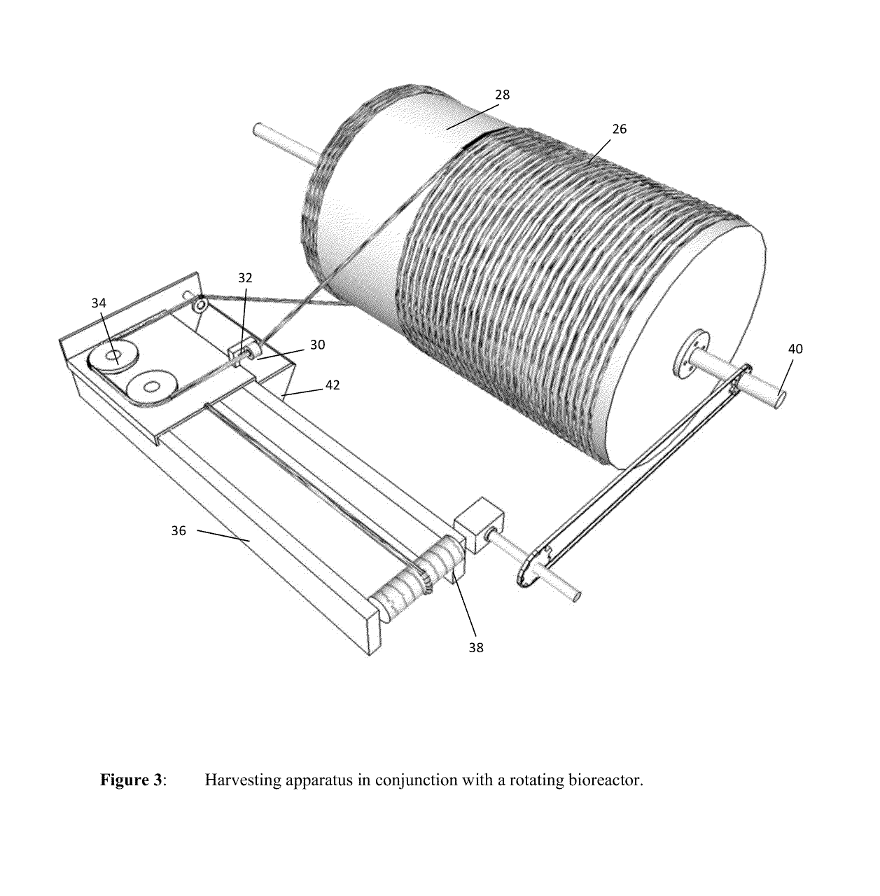

In another embodiment, a harvesting apparatus in conjunction with a rotating bioreactor may be employed. Referring now to FIG. 3, the biofilm is collected by detaching one end of the substratum 26 from the cylinder 28 and threading it through a scraper 30. The scraper 30 may be a blade, series of blades, simple piece of rigid material with a hole in it, or more preferably, a unit with an adjustable diameter and/or constant tension settings like a hose clamp. The scraper 30 may be held in place by attachment to a support 32. A reorientation system 34 is provided to prevent twisting or binding of the substratum 26. The loose end of the substratum 26 is threaded through the scraper 30 and reorientation system 34 until it can be reattached to the cylinder 28. As the cylinder 28 continues to rotate, the entire length of the substratum 26 may be pulled through the scraper 30 and pulley system 34 and rewound onto the cylinder 28. To ensure the substratum 26 may be properly rewound onto the entire length of the cylinder 28, the scraper 30, support 32, and pulley system 34, are pulled on a support frame 36 along the length of the cylinder 28 at a rate such that the harvested portion of the substratum 26 may not be layered on top of itself as it is rewound. This may be accomplished with a lateral movement system 38 that may be powered by connection to the drive shaft 40 powering the cylinder 28. Appropriate gear ratios may be chosen to achieve the desired pull rate and spacing of substratum 26. As the biofilm is removed from the substratum 26, it is gathered in a collection bin 42.

Referring now to another embodiment describing a multiple cylinder setup, shown in FIG. 4, a drive shaft 44 may be made long enough to support two or more cylinders 46 in a train formation. More cylinders 46 may be placed so that rotational power from a motor 48 is transferred to two or more drive shafts 44 through a power transfer mechanism like a roller chain 50. The drive shafts 44 are supported by bearings 52 on each end.

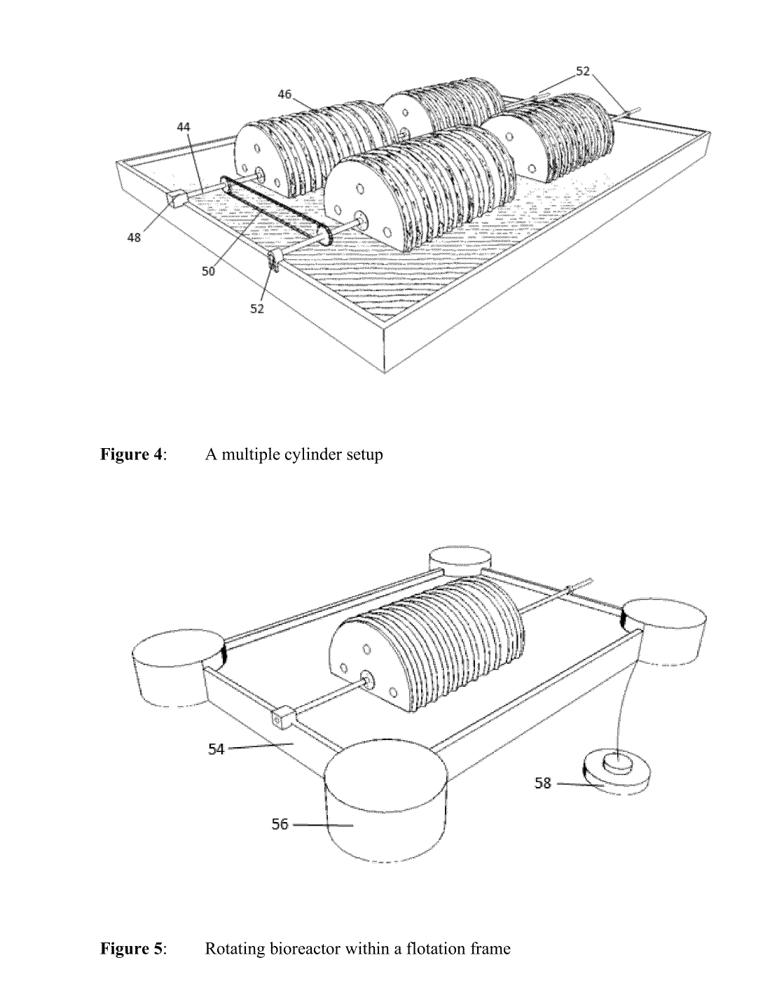

Referring to another embodiment shown in FIG. 5, the entire apparatus may be placed within a support frame 54 with attached floats 56. The apparatus can then be placed at a suitable site and held in place using an anchor 58 or other suitable means of holding it in place. One application of this embodiment of the invention is a retrofitting of oxidation lagoons at a wastewater treatment plant.

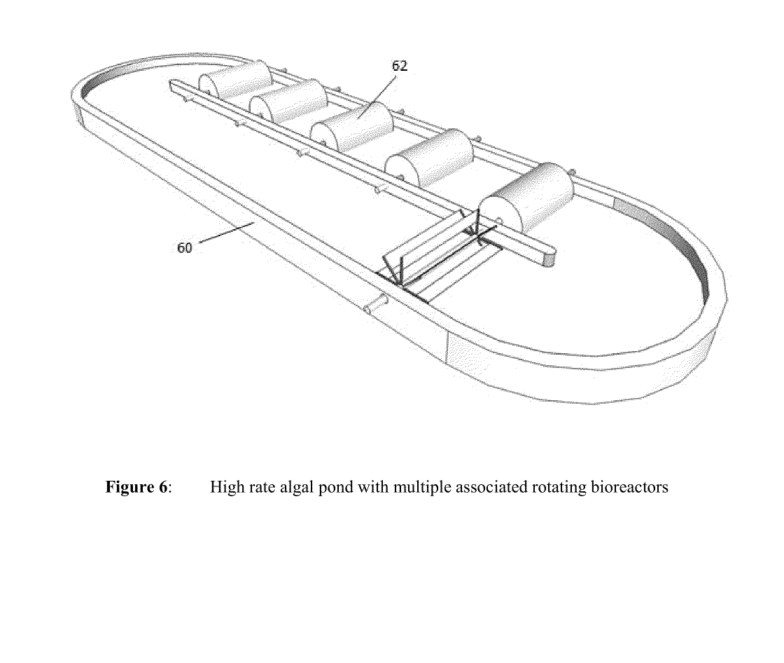

Referring to FIG. 6, another embodiment places the apparatus with a high rate algae pond 60 like a raceway or meandering ditch. The cylinders 62 may be rotated by the force of the passing water or powered by a motor and shaft connected to the cylinder. In a further embodiment, the cylinders 62 may be rotated by an air supply directed at the submerged perimeter of the cylinder in a direction perpendicular to the axis of rotation. In embodiments such as this, the biofilm enhances flocculation of the suspended culture, leading to inexpensive harvesting of all the biomass in the system.

### Example I.A.1

In one embodiment, several bench scale units of the type shown in FIG. 2 were used with 8 liters of chlorinated weak domestic strength wastewater as seeding media. A nested factorial experiment with triplicate replication of samples was established to determine the most suitable substrata for biofilm growth. The initial total suspended solids content of the wastewater was 42 mg/l. Concentrations of soluble phosphorus and nitrogen were brought to 5 mg/l and 36 mg/l respectively using KH₂PO₄, K₂HPO₄, and NaNO₃. As a fed batch operation, N and P were added every 48 hours to give an average total P of 5.0 mg/l, and an average total N of 52.7 mg/l. Soluble N and P averaged 26.2 mg/l and 3.7 mg/l, respectively. A light cycle of 14 hours on, 10 hours off was used throughout the experiment.

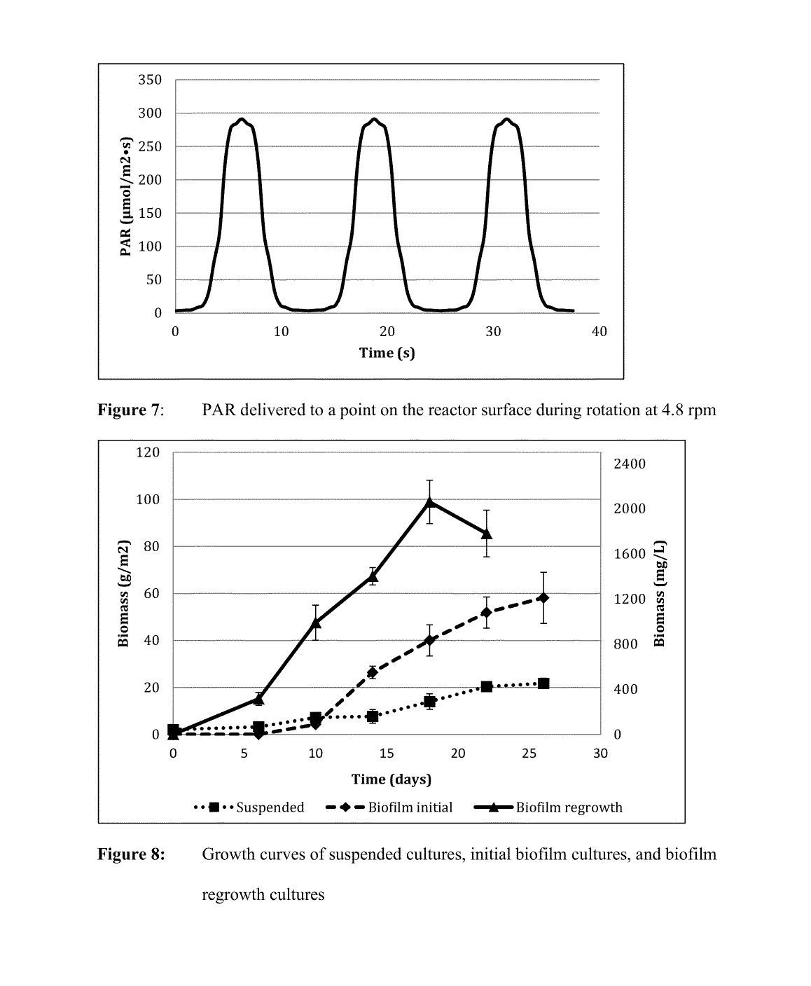

FIG. 7 shows the cycle of photosynthetically active radiation (PAR) delivered to a point on the reactor during rotation at 4.8 rpm during periods while the lights were on. Biomass was harvested after 22 days of growth. This time included a recovery period following chlorination. Table 1 summarizes the results on the basis of mass per liquid surface area.

TABLE 1
Avg. Biomass yield of different substrate materials

| | Avg. Biomass Yield | |
|---|---|---|
| Substrata | (g/m²) | Std. Deviation |
| Cotton Rope | 91.2 | 10.4 |
| Cotton (High thread count) | 62.2 | 0.9 |
| Jute | 51.4 | 5.1 |
| Cotton (Low thread count) | 51.3 | 1.9 |
| Acrylic | 49.3 | 0.4 |
| Polyester | 19.3 | 1.8 |
| Polypropylene | 0 | 0 |
| Nylon | 0 | 0 |
| Construction Paper* | 0 | N/A |
| Sisal* | 0 | N/A |
| Lignin based cover* | 0 | N/A |

*Materials showed some growth but biomass was not harvestable

The substrata that were placed onto the cylinder as a sheet were harvested using a simple scraper blade. This proved to be difficult due to the constant adjustments required to scrape the uneven biofilm growth. Such substrata had also loosened during reactor operation causing frequent snagging and tearing against the scraper blade and rendering the materials unsuitable for future use. Cotton rope gave the highest biomass yields, and the rope construction allowed application of the harvesting method shown in FIG. 3. The cotton rope incurred no damage during harvesting and was immediately reused.

### Example I.A.2

In another embodiment, the same procedure described in Example I.A.1 was repeated with cotton rope as the only substratum. Triplicate samples were harvested after 10, 14, 18, 22, and 26 days of growth. Suspended cultures were also grown in reactor tanks of the same dimensions with the same light and nutrient conditions as the biofilm reactors. The same weak domestic strength wastewater was used to seed each type of reactor. Power input for mixing the suspended cultures was the same as the power input for rotating the cylinders. After each biofilm harvest, the substrata were reloaded onto the reactor to determine the secondary growth curve. Regrowth samples were harvested after 6, 10, 14, 18, and 22 days of growth. Growth in the suspended culture reactors was determined using the glass fiber filter method.

FIG. 8 shows the growth curves of the initial biofilms, secondary biofilms, and suspended cultures. It can be seen that the biofilm grows at a much faster rate after the initial harvest. This is most likely due to the residual biomass left on the substratum after scraping. This secondary growth curve represents the true productivity of the reactor when operated continuously. Table 2 compares the maximum productivity obtained by each type of growth and at what time it was obtained.

TABLE 2
Maximum productivity obtained by different growth types

| | Yield* | Time | Productivity* |
|---|---|---|---|
| Growth Type | (g/m²) | (days) | (g/m² day) |
| Biofilm initial | 51.6 ± 6.6 | 22 | 2.4 ± 0.3 |
| Biofilm regrowth | 98.9 ± 9.3 | 18 | 5.5 ± 0.5 |
| Suspended | 20.4 ± 1.4 | 22 | 0.9 ± 0.1 |

*plus and minus one standard deviation from the mean

### Example I.A.3

In another embodiment, nitrogen and phosphorus concentration data from the experiment of Example I.A.2 were analyzed to determine the wastewater remediation ability of the suspended culture and the biofilms. After filtration of wastewater samples, soluble N concentrations were determined using the chromotropic acid method for nitrate-N and the salicylate method for ammonia-N. Soluble P as orthophosphate was determined using the ascorbic acid method. The wastewater samples were also analyzed for total N and P using the chromotropic acid method with alkaline persulfate digestion and the molybdovanadate method with acid persulfate digestion, respectively.

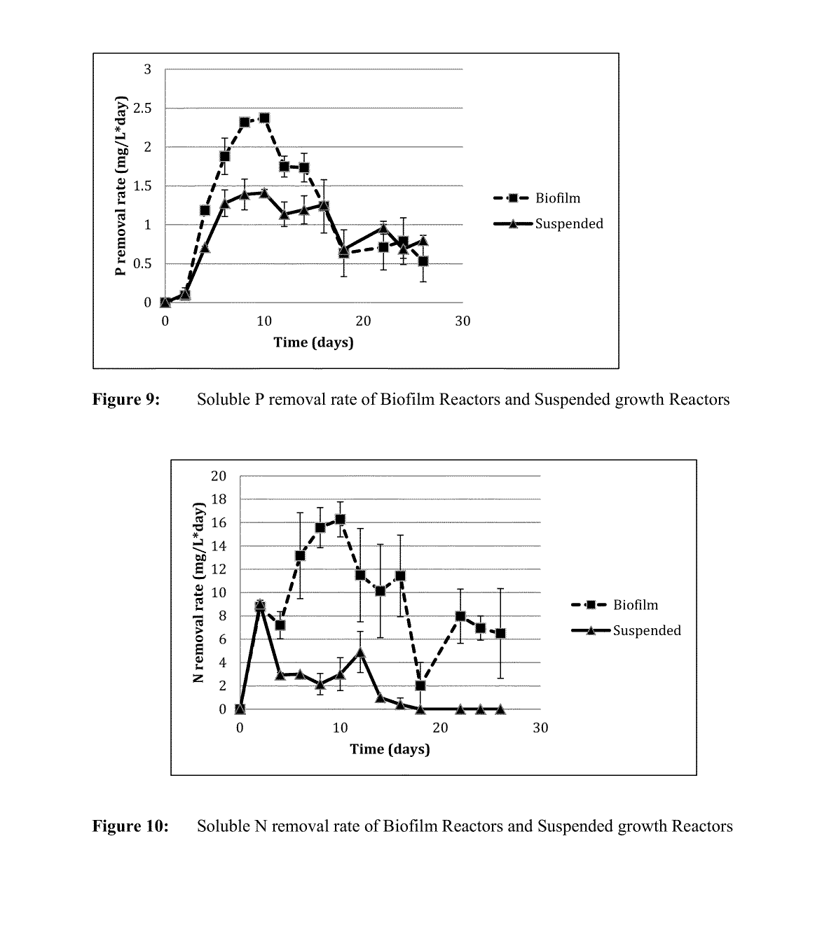

FIG. 9 shows soluble P removal rates for the suspended and biofilm cultures. FIG. 10 shows soluble N removal rates for the suspended and biofilm cultures. It can be seen that the biofilm reactors demonstrated higher removal of both nitrogen and phosphorus compared to the suspended culture reactors. Furthermore, these nutrients could be easily removed from the system by simply removing the biofilm as shown in FIG. 3, whereas the suspended cultures would have to be removed through centrifugation, filtration, or the like to completely remove the nutrients from the system.

### Example I.A.4

In another embodiment, as the biofilms of the experiments of Example I.A.1 and Example I.A.2 were grown, a visual observation of the wastewater turbidity was made for each tank containing a rotating bioreactor. It was observed that at some point during operation, typically between 12-18 days of growth, the suspended microorganisms in the wastewater associated with the rotating bioreactors underwent spontaneous auto-flocculation and settled to the bottom or floated to the top of the reactor tank. Such flocculated biomass would be much easier to harvest than a suspended culture.

B. Organic Coagulants and Flocculants

In embodiments, organic coagulants and flocculants may be employed to effectively harvest algae without negatively affecting the various bio-products that may be later derived from algae. Exemplary bio-products of algae include bio-plastics, biodiesel, biosolvents, and numerous other products. In embodiments, the organic coagulant and flocculant may comprise a modified starch as described herein or as described in U.S. Patent Application No. 61/552,604, filed Oct. 28, 2011 (the entirety of which is herein incorporated by reference).

(1) Starch

Starch is an abundant natural polymer available from sources such as potato, corn, rice, tapioca, etc. Irrespective of the source, starch is primarily comprised of amylose (20-30% wt) and amylopectin (70-80% wt), which are illustrated below:

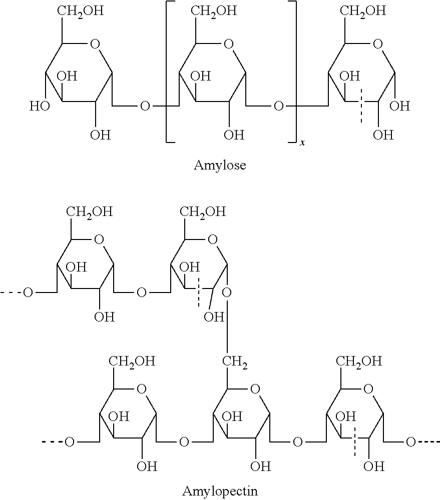

In some embodiments, the starch source may be what would otherwise be considered a waste product, such as waste starch derived from potato, or other vegetable, processing.

The starch may be modified to have cationic groups, such as amine, ammonium, phosphonium, or imines. By modifying the starch with cationic groups, the starch may then serve as an organic coagulant and flocculant for algae harvesting.

(2) Starch Modification

The starch may be modified by any suitable method. In some embodiments, the starch is modified by initiating free radicals on the starch backbone and grafting a quaternary ammonium moiety on to it, as set forth in the following reaction scheme:

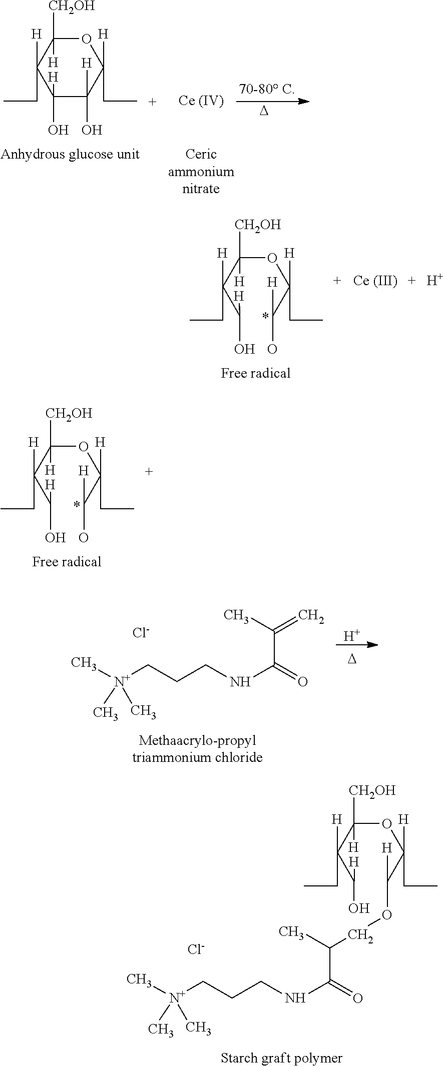

The free radical generation and quaternary ammonium can be achieved through other chemicals and reagents. For example, for the free radical generation ferrous ion-peroxide or potassium persulfate/sodium thiosulfate redox system can be used and [2-(Methacryloyloxy)-ethyl]-trimethylammoniumchloride (TMAEMA) or [3-(Methacryloylamino)-propyl]-trimethylammoniumchloride (MAPTAC) or Diallyldimethylammoniumchloride (DADMAC) can be used as quaternary ammonium.

To begin with, free radicals on the starch backbone (e.g. corn or potato) can be generated by addition of ceric ammonium nitrate to a gelatinized starch mixture at 60-90° C. for 15-60 minutes. After generating free radicals, [3-(Methacryloylamino)-propyl]-trimethylammoniumchloride (MAPTAC) is added and the mixture is made acidic to pH 2-4 by the addition of nitric acid. The mixture is heated for 2-6 hours at 60-90° C. after which it is allowed to slowly cool to room temperature.

This modified starch may be separated from the solution by precipitation with, for example, ethanol. The solution may be centrifuged, or otherwise subjected to a solid-liquid separation technique, to collect the precipitate and the supernatant may then be discarded. The precipitate may be washed with a suitable washing agent, such as ethanol in a soxhlet apparatus with a reflux time which may include up to 20 hours, such as about 5 to 15 hours, or about 12 hours to clean the starch of any unreacted reagents and catalyst. The modified starch may then be dried of the washing agent, optionally pulverized, and stored at room temperature until further use.

After modified starch preparation, the zeta potential may be measured to examine the potency of the modified starch as a potential coagulant and flocculant. Zeta potential is the measure of charge present on a colloidal particle surface. For the modified starch to show cationization, the zeta potential should be greater than 0. Minimum zeta potential above about +1 mV is necessary for the feasibility of starch as a coagulant/flocculant for algae separation and harvesting. Suitable zeta potentials for the modified starch as a coagulant/flocculant may include, for example, from about +5 to about +20 mV in a pH of about 5.0 to about 10.0.

Degree of substitution (DS) relates to the number of hydroxyl groups (maximum 3) that are substituted by quaternary ammonium. In embodiments, the higher the degree of substitution, the greater would be the neutralizing capability of a modified starch resulting in efficient separation with minimal dosage. Suitable DS values may include, for example, from about 0.82 to about 1.34.

(3) Precipitate Formation

The CAS, or modified starch, may be mixed with an aqueous solution containing algae to be harvested. Suitable ratios include, for example, from about 0.5:1.0 to 3.0:1.0 starch:algae. Upon addition of the modified starch, the solution may be optionally flash mixed to facilitate uniform mixing of the modified starch in the suspension for charge neutralization and to avoid lump formation. Flash mixing may be followed by slow mixing to facilitate bridging (particle interaction between algae and starch) of the neutralized algae particles and also to help in residual charge neutralization not achieved by flash mixing. The mixing may be then stopped and the flocs are allowed to sediment for a period of time. Precipitate formation may be performed in a suitable reactor equipped with optional stirrers and/or convection properties.

The following examples are illustrative only and are not intended to limit the disclosure in any way.

### Example I.B.1

Preparation of cationic starch graft polymer. In the preparation of cationic starch graft polymer, the first step was to generate free radicals on the starch backbone by dissolving 5 grams of starch (corn or potato) in 100 ml di-ionized water at 75° C. for 30 minutes. To this starch slurry, 0.5 g of ceric ammonium nitrate ((NH₄)₂Ce(NO₃)₆) was added slowly and allowed to dissolve completely at 75° C. for 30 minutes. For grafting of quaternary ammonium, 15 ml of [3-(Methacryloylamino)-propyl]-trimethylammoniumchloride (MAPTAC) (50% in water) was added slowly by continuous stirring. The pH of the mixture was adjusted to pH 3 by the addition of nitric acid (HNO₃) and the polymerization reaction was allowed to proceed for 2 hours. After the specified reaction time, the mixture was allowed to cool to room temperature and the pH was neutralized to pH 7.0 by the addition of hydrochloric acid (HCl). The starch was precipitated out of solution by the addition of ethanol as needed. The solution was centrifuged at 8000 rpm for 5 mins after which the supernatant was discarded. The recovered starch was washed in a soxhlet apparatus with ethanol for 8 hours to clean the starch of any unreacted chemicals or reagents. The washed starch was dried, pulverised and stored until further use.

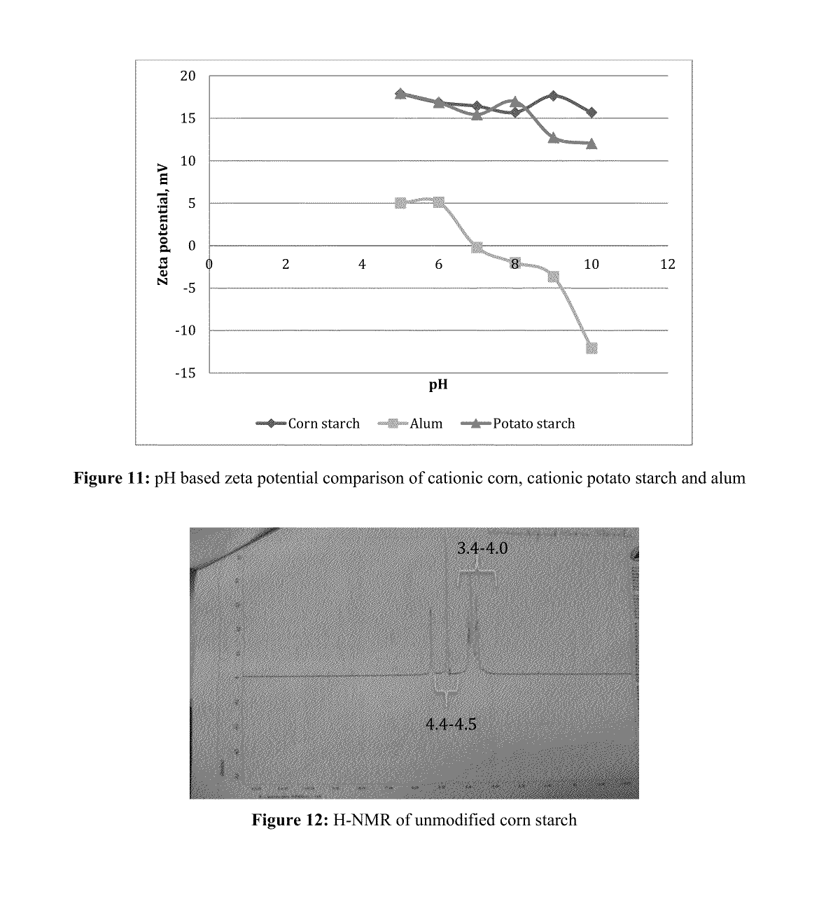

The zeta potential for cationic starch graft polymer was measured across a varying pH range (5 to 10). This experiment illustrates the difference in zeta potential behavior with varying pH for cationic starch graft polymer. As is illustrated in FIG. 11, as the pH increased, the zeta potential of cationic starch graft polymer stays nearly constant on the positive region, average +16 mV and +15 mV for corn and potato starch, respectively due to the effect of quaternary ammonium on the starch molecule which shows independence of pH in terms of zeta potential.

The total nitrogen content of the cationic starch graft polymers was determined using a Hatch Total N kit in order to determine the degree of substitution. The degree of substitution was calculated using the following formula:

$$Degree of substitution , DS = \frac{161 \times N \%}{[ 1400 - ( 220.74 \times N \% ) ]}$$

161=M.W. of starch; 220.74=M.W. of MAPTAC; % N=% of total N in starch. The degree of substitution is a measure of substitution of the hydroxyl group in one anhydrous glucose unit of starch. One anhydrous glucose unit of starch contains 3 hydroxyl groups. Hence, the maximum degree of substitution that a modified starch can attain is 3. The test revealed DS of 1.34 and 0.82 for corn and potato cationic starch graft polymers, respectively. This test confirms the attachment of MAPTAC to the starch molecule in the cationic starch graft polymer

H-NMR

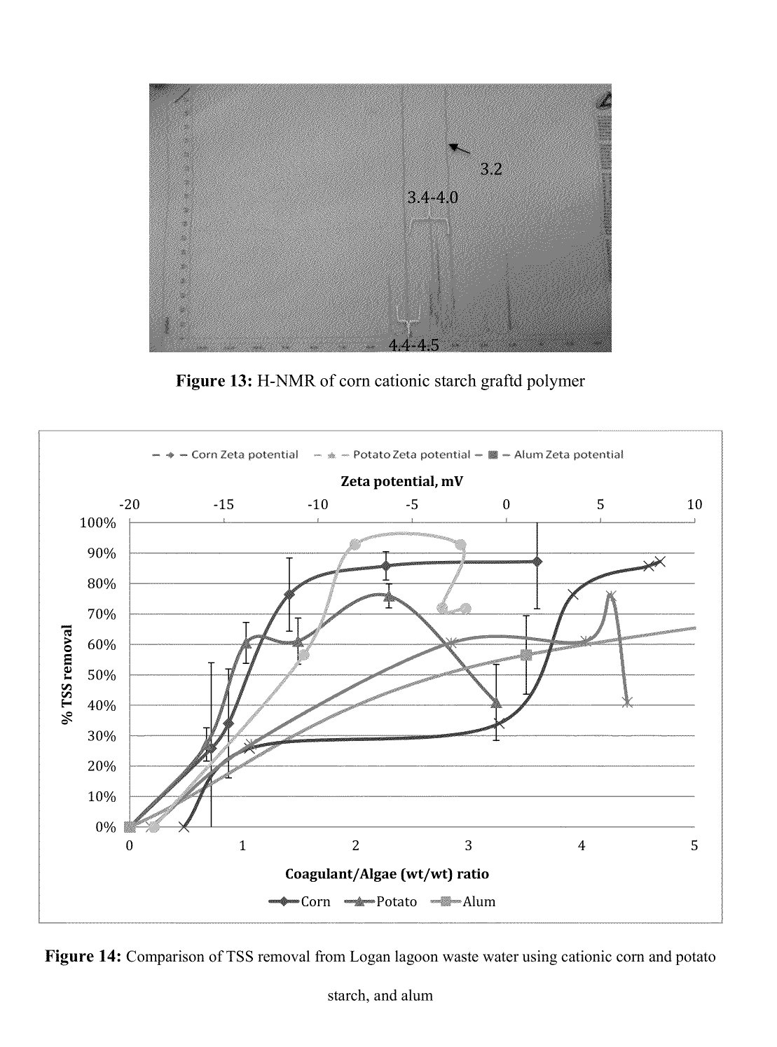

Proton NMR analysis was performed on unmodified and corn cationic starch graft polymer. The H-NMR spectra shown on FIG. 12 represents unmodified corn starch. The peak at 4.4-4.5 ppm is attributed to the proton associated with C-1 carbon on the anhydrous glucose unit (AGU) of starch. The peaks from 3.4-4.0 ppm are attributed to the other protons on the AGU. The strong peak in FIG. 13 at 3.2 ppm is attributed to the protons surrounding the nitrogen atom attached to the starch molecule.

### Example I.B.2

Jar Test. Jar test were performed to optimize the dosages of the cationic starch graft polymers and to compare the coagulation/flocculation efficiencies with that of aluminum sulfate (Al₂(SO₄)₃. H₂0) (Alum) using wastewater from the Logan lagoons at an average initial concentration of 50 mg/L.

Jar tests were performed in triplicate for each of the coagulant/flocculants i.e. corn cationic starch graft polymer, potato cationic starch graft polymer and alum. Total suspended solid (TSS), zeta potential and total phosphorus (TP) were the parameters that were measured of the wastewater before and after addition of the coagulants/flocculants. Total suspended solids were measured using 2540 D Standard Methods. Zeta potential was measured using Brookhaven ZetaPlus zeta meter. Total phosphorus was measured using Lachat 8500 QuikChem.

Cationic corn starch showed TSS removal of about 80% with a coagulant/algae weight ratio of 1.4. Cationic potato starch on the other hand showed 60% TSS for the same ratio. The flocculation behavior of the cationic starches was observed with lagoon algae as well. A slight change in zeta potential of the colloids resulted in significant TSS removal efficiencies. The cationic starches showed high potency as coagulant/flocculant with high TSS removal efficiency when compared to alum which shows only about 30% TSS removal with the same coagulant/algae ratio. A significantly higher coagulant/algae ratio of 3.5 was required for alum to effect 63% TSS removal. The results are illustrated in FIG. 14

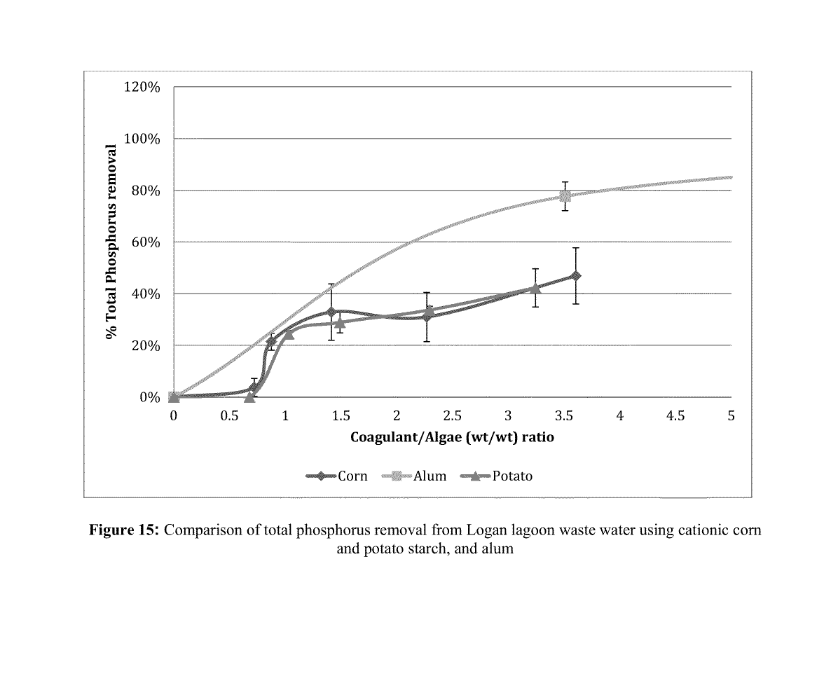

Total phosphorus (TP) removal tests were performed alongside TSS removal for the wastewater. Initial concentrations of TP in the Logan lagoons wastewater ranged from 3.0 to 4.0 mg/L. Total phosphorus comprises of soluble and insoluble phosphorus. The insoluble phosphorus comprises of algae or TSS and is taken out of solution with the TSS. Soluble phosphorus comprises of orthophosphate. FIG. 14 shows the total phosphorus removal efficiency of cationic corn and potato starch, and alum tested on the wastewater from the Logan lagoons. Cationic corn starch shows about 33% and potato starch shows about 29% TP removal associated with TSS %. Alum shows about 42% TP removal and when compared to TSS removal indicates simultaneous TP and TSS removal mechanism taking place. The TP removal graph for cationic corn and potato starch shows an upward trend after ratios of 3-3.5. The coagulant/algae ratio of 3-3.5 is when TSS removal for the respective starches reaches a maximum. This suggests a stepwise TSS and then Total phosphorus removal as opposed to alum. The trends show a higher dosage of cationic corn and potato starch would achieve precipitation of the soluble orthophosphate. The results are illustrated in FIG. 15.

### Example I.B.5

Algae harvesting methodology for processing. Two algal cultures namely, microalga Scenedesmus obliquus and lagoon wastewater was used for harvesting algae from. The coagulants/flocculants used were potato cationic starch graft polymer and alum. Algae from these cultures were also harvested by centrifugation in order to serve as a control for processing.

The basis of algae harvesting with coagulants was to reduce the negative zeta potential on the algae in the cultures to 0 mV. This makes the cultures destabilized and the algae precipitates out. This method was chosen in order to normalize the differences in dosage of potato cationic starch and alum on the different algal cultures. After charge neutralization, the algal precipitate was separated by gravity settling for an hour after which the supernatant was disposed and the precipitate was further concentrated by centrifugation. The precipitate was freeze dried and a small sample was washed several times in slightly basic aqueous solution to obtain actual algae weight in the precipitate. The following table summarizes the total mass (algae+coagulants/flocculants) collected and the % of the actual algae dry weight in total mass.

LOGAN LAGOON ALGAE

| Harvesting method | Total weight, grams | % dry wt of algae |
|---|---|---|
| Centrifuged | 10 | 100 |
| Cationic Potato starch | 8.6 | 93 |
| Alum | 25.5 | 55 |

### II. Processing Biomass

Upon harvest or acquisition of biomass, the biomass may be processed as described below. In systems described herein, collected biomass may be initially processed with a wet lipid extraction procedure. After such a procedure, the various intermediary products may be further processed into various bioproducts, such as biofuels, biosolvents, and bioplastics, each of which are described below in more detail.

A. Wet Lipid Extraction Procedure

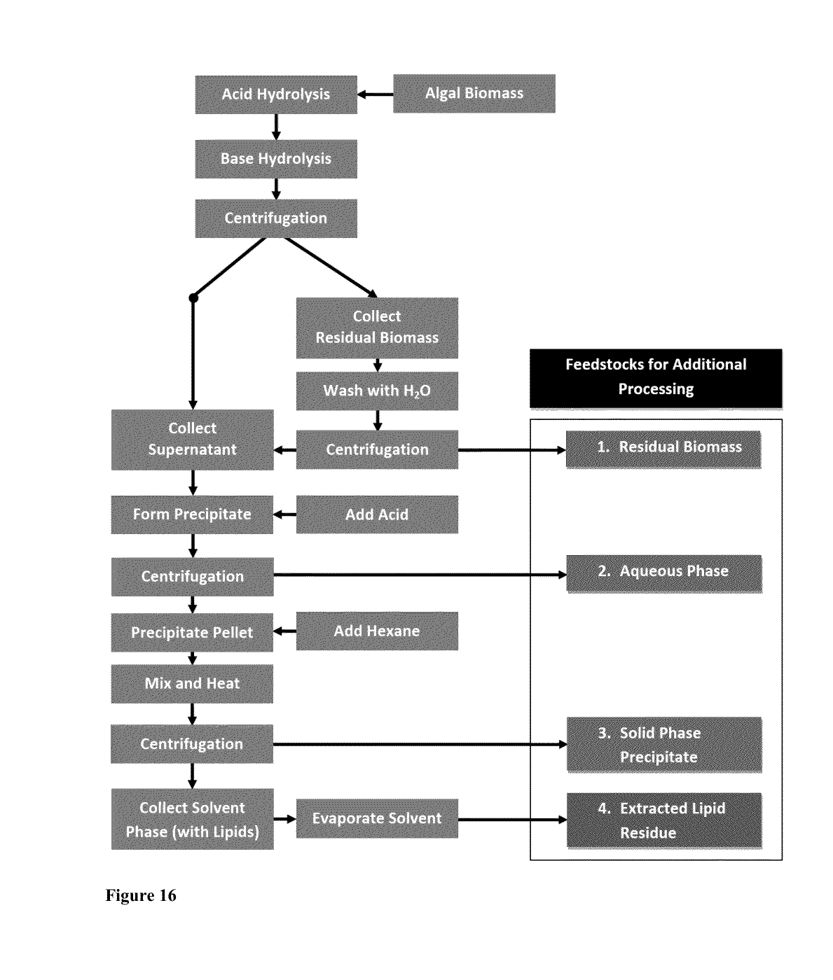

In some embodiments, the system may include Wet Lipid Extraction Procedure (“WLEP”), as described in U.S. Application No. 61/551,049, filed Oct. 25, 2011, the entirety of which is herein incorporated by reference in its entirety. WLEP may include the following steps: (1) acid hydrolysis, (2) base hydrolysis, (3) biomass and aqueous phase separation, (4) precipitate formation, and (5) free fatty acid extraction. FIG. 16 illustrates a flow diagram of an exemplary method.

Feed Stock

As a feed stock, any suitable biomass may be used. In embodiments, algae that produces high lipid amounts may be preferred. In many embodiments, algae produced on waste water may be used. The algae may be lyophilized, dried, in a slurry, or in a paste (with for example 10-15% solid content). In the system described herein, the biomass may be harvested according to the harvesting processes described above.

After identification of a feed stock source or sources, abiomass, such as algae, may be formed into a slurry, for example, by adding water, adding dried or lyophilized algae, or by partially drying, so that it has a solid content of about 1-40%, such as about 4-25%, about 5-15%, about 7-12%, or about 10%.

The various steps to the process, according to some embodiments, is described in more detail below. The methods described herein may be accomplished in batch processes or continuous processes.

(1) Acid Hydrolysis

To degrade the algal cells (or other cells present), to bring cellular components into solution, and to break down complex lipids to free fatty acids, the slurry of water and algae described above may be optionally heated and hydrolyzed with at least one acidic hydrolyzing agent. These complex lipids may include, for example, triacylglycerols (TAGs), phospholipids, etc. In addition to degrading algal cells and complex lipids, the acidic environment created by addition of the hydrolyzing agent removes the magnesium from the chlorophyll molecules (magnesium can otherwise be an undesirable contaminant in end-product biodiesel).

When heated, the slurry may reach temperatures of from about 1-200° C., such as about 20-100° C., about 50-95° C., or about 90° C. When temperatures above 100° C., or the boiling point of the solution are used, an apparatus capable of withstanding pressures above atmospheric pressure may be employed. In some embodiments, depending on the type of algae, the type and concentration of acid used for hydrolysis, the outside temperature conditions, the permissible reaction time, and the conditions of the slurry, heating may be omitted. Heating may occur prior to, during, or after addition of a hydrolyzing agent.

In addition, the slurry may be optionally mixed either continuously or intermittently. Alternatively, a hydrolysis reaction vessel may be configured to mix the slurry by convection as the mixture is heated.

Acid hydrolysis may be permitted to take place for a suitable period of time depending on the temperature of the slurry and the concentration of the hydrolyzing agent. For example, the reaction may take place for up to 72 hours, such as from about 12-24 hours. If the slurry is heated, then hydrolysis may occur at a faster rate, such as from about 15-120 minutes, 30-90 minutes, or about 30 minutes.

Hydrolysis of the algal cells may be achieved by adding to the slurry a hydrolyzing agent, such as an acid. Any suitable hydrolyzing agent, or combination of agents, capable of lysing the cells and breaking down complex lipids may be used. Exemplary hydrolyzing acids may include strong acids, mineral acids, or organic acids, such as sulfuric, hydrochloric, phosphoric, or nitric acid. These acids are all capable of accomplishing the goals stated above. When using an acid, the pH of the slurry should be less than 7, such as from about 1-6, about 1.5-4, or about 2-2.5.

In addition to strong acids this digestion may also be accomplished using enzymes alone or in combination with acids that can break down plant material. However, any such enzymes or enzyme/acid combinations would also be capable of breaking down the complex lipids to free fatty acids.

In some embodiments, the acid or enzymes, or a combination thereof, may be mixed with water to form a hydrolyzing solution. However, in other embodiments, the hydrolyzing agent may be directly added to the slurry.

(2) Base Hydrolysis

After the initial hydrolysis, a secondary base hydrolysis may be performed to digest and break down any remaining whole algae cells; hydrolyze any remaining complex lipids and bring those lipids into solution; convert all free fatty acids to their salt form, or soaps; and to break chlorophyll molecules apart.

In this secondary hydrolysis, the biomass in the slurry is mixed with a basic hydrolyzing agent to yield a pH of greater than 7, such as about 8-14, about 11-13, or about 12-12.5. Any suitable base may be used to increase in pH, for example, sodium hydroxide, or other strong base, such as potassium hydroxide may be used. Temperature, time, and pH may be varied to achieve more efficient digestion.

This basic slurry may be optionally heated. When heated, the slurry may reach temperatures of from about 1-200° C., such as about 20-100° C., about 50-95° C., or about 90° C. When temperatures above 100° C., or the boiling point of the solution are used, an apparatus capable of withstanding pressures above atmospheric pressure may be employed. In some embodiments, depending on the type of algae, the type and concentration of acid used for hydrolysis, the outside temperature conditions, the permissible reaction time, and the conditions of the slurry, heating may be omitted. Heating may occur prior to, during, or after addition of a hydrolyzing agent.

In addition, the basic slurry may be optionally mixed either continuously or intermittently. Alternatively, a hydrolysis reaction vessel may be configured to mix the slurry by convection as the mixture is heated.

Basic hydrolysis may be permitted to take place for a suitable period of time depending on the temperature of the slurry and the concentration of the hydrolyzing agent. For example, the reaction may take place for up to 72 hours, such as from about 12-24 hours. If the slurry is heated, then hydrolysis may occur at a faster rate, such as from about 15-120 minutes, 30-90 minutes, or about 30 minutes.

During this basic and/or acid hydrolysis, chlorophyll is hydrolyzed to the porphyrin head and phytol side chain.

(3) Biomass and Aqueous Phase Separation

Under the condition of elevated pH, the biomass may be separated from the aqueous solution. This separation is performed while the pH remains high to keep the lipids in their soap form so that they are more soluble in water, thereby remaining in the aqueous phase. Once the separation is complete, the aqueous phase is kept separate and the remaining biomass may be optionally washed with water to help remove any residual soap molecules. This wash water may also be collected along with the original liquid phase. Once the biomass is washed it may be removed from the process.

The aqueous phase now contains the recovered lipids in soap form, Porphyrin salts, and any other soluble cellular components. Much of the hydrophobic or insoluble cellular components are potentially removed with the biomass.

Any suitable separation technique may be used to separate the liquid (aqueous) phase form the biomass. For example, centrifugation, gravity sedimentation, filtration, or any other form of solid/liquid separation may be employed.

(4) Precipitate Formation

After the biomass is removed, the pH of the collected liquid may be neutralized/reduced to form a precipitate. This may be accomplished by the addition of an acid to the solution, such as at least one strong acid or mineral acid, for example, sulfuric, hydrochloric, phosphoric, or nitric acid. Addition of a suitable acid is performed until a green precipitate is formed. The green precipitate may contain, or may be, the Porphyrin heads as they are converted from their salt forms. It may also contain proteins and other cellular components that are coming out of solution.

The pH may be reduced to a pH of about 7 or less, such as about 4-6.9. This lower pH also converts the soap in the liquid to free fatty acids. As the precipitate forms the fatty acids associate with the solid phase and come out of solution. Once the precipitate has formed, the solid and liquid phases may be separated. Any suitable separation method may be employed, such as centrifugation, gravity sedimentation, filtration, or any other form of solid/liquid separation. The liquid phase may be removed from the process. The collected solid phase may then be processed further. Optionally, the precipitate may be lyophilized or dried, which may result in nearly complete extraction of the lipids during extraction.

(5) Free Fatty Extraction and Solvent Recycle

To extract the free fatty acids, an organic solvent may be added to the solid phase resulting from the previous step. The solid phase may be mixed with the solvent and then optionally heated to facilitate fatty acid extraction from the solid phase.

When heated, the mixture of solid phase and solvent may reach temperatures of from about 1-200° C., such as about 20-100° C., about 50-9° C., or about 90° C. When temperatures above 100° C., or the boiling point of the solution are used, an apparatus capable of withstanding pressures above atmospheric pressure may be employed. In some embodiments, heating may be omitted. Heating may occur prior to, during, or after the mixture of solid phase and solvent is formed. In addition, the mixture may be optionally mixed either continuously or intermittently.

The extraction process may be permitted to take place for a suitable period of time depending on the temperature of the mixture. For example, the extraction may take place for up to 72 hours, such as from about 12-24 hours. If the mixture is heated, then extraction may occur at a faster rate, such as from about 15-120 minutes, 30-90 minutes, or about 30 minutes.

During this time the free fatty acids associated with the solid are extracted into the organic phase. Suitable solvents include non-polar solvents, such as hexane, chloroform, pentane, tetrahydrofuran, and mixtures thereof (for example a 1:1:1 (v/v) ratio of chloroform, tetrahydrofuran, and hexane). Other suitable solid-liquid extraction methods and unit operations may be used.

Once the free fatty acids are extracted, the solid phase may be removed from the process and the organic phase may be vaporized and recycled. What remains after the organic phase is vaporized is a residue containing free fatty acids or algal lipids/oil. This algal oil may then optionally be processed into biodiesel.

The following examples are illustrative only and are not intended to limit the disclosure in any way.

### Example II.A.1

Acid Hydrolysis. To a glass test tube 100 mg of lyophilized algal biomass was added. One mL of a 1 Molar Sulfuric acid solution is added to the test tube and the test tube was then sealed using a PTFE lined screw cap and gently mixed to create a homogenous slurry. This slurry was then placed in a Hach DRB-200 heat block pre-heated to 90° C. This slurry is allowed to digest for 30 minutes with mixing at the 15 minute mark.

### Example II.A.2

Base Hydrolysis. Once the first 30 minute digestion period of Example II.A.1 was complete, the test tube was removed from the heat source and 1.0 mL of a 5 Molar Sodium Hydroxide solution was added to the test tube. The test tube was immediately resealed and returned to the heat source for 30 minutes. Mixing at 15 minutes was again provided.

### Example II.A.3

Biomass Removal. Once the base hydrolysis step of Example II.A.2 was complete, the test tube was removed from the heat source and allowed to cool in a cold water bath. Once cooled the test slurry was centrifuged using a Fisher Scientific Centrific Model 228 centrifuge. The upper aqueous phase was removed and collected in a separate test tube. To the remaining biomass 1 mL of deionized water as added and vigorously mixed. The slurry was re-centrifuged, and the liquid phase collected and added to the previously collected liquid phase. The biomass was then removed from the process.

### Example II.A.4

Precipitate Formation. To the collected liquid phase of Example II.A.3, 3 mL of a 0.5 Molar Sulfuric Acid Solution was added, or until a green precipitate was formed. After mixing the liquid became a solid-liquid slurry. This mixture was centrifuged and the upper aqueous phase was removed from the process and the solids were further processed.

### Example II.A.5

Free Fatty Acid Extraction. Five milliliters of Hexane was added to the collected precipitate of Example II.A.4, which was sealed using a PTFE lined screw cap, and vigorously mixed. The test tube was then placed in the Hach DRB-200 heat block, pre-heated to 90° C. Extraction of the free fatty acids into the Hexane phase was allowed to continue at 90° C. with mixing provided every 5 minutes. After a time duration of 15 minutes at 90° C. was completed, the test tube was centrifuged to pellet the solids and to allow for the collection of the solvent phase, which as transferred to another test tube. Hexane was allowed to vaporize via gentle heating within the test tube leaving behind the free fatty acid residue.

### Example II.A.6

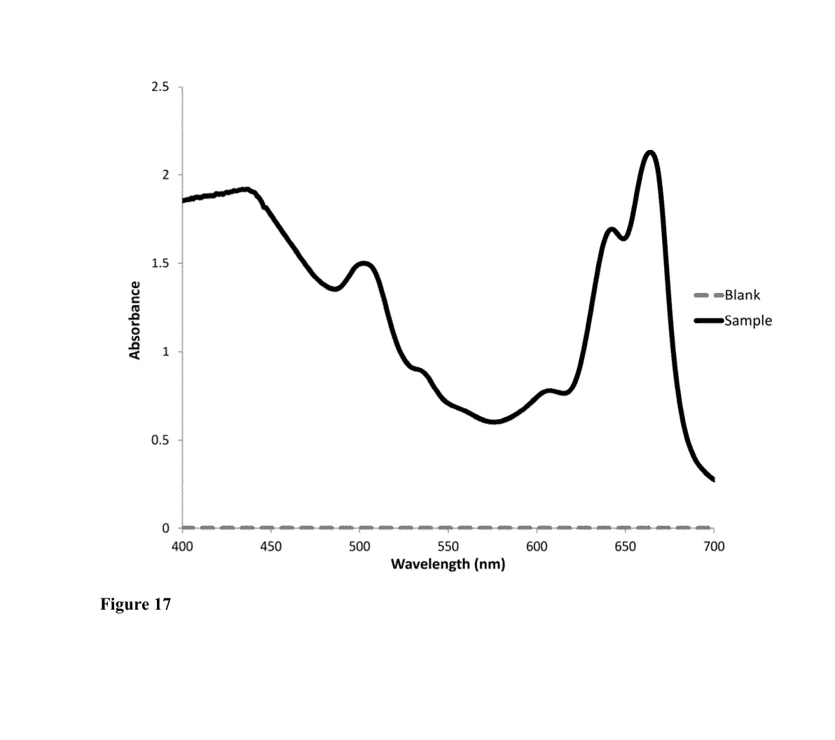

Pigment Precipitation. The process outlined in Examples II.A.1-4 was performed on a sample. The resulting precipitate was freeze-dried and then re-dissolved in 5 M sodium hydroxide. The resulting solution was analyzed using a Shimadzu UV-1800 UV Spectrophotometer. The slide showed absorption data from a Shimadzu UV-1800 UV Spectrophotometer, which measures the absorbance from 300 nm to 900 nm. The results are shown in FIG. 17. The “blank,” or lower line along the bottom, refers to plain 5 M Sodium Hydroxide; and the “sample” refers to the re-dissolved precipitate. The spectrum resulting from the analysis of the precipitate showed strong absorbance at wavelengths typical of chlorophyll. The data developed demonstrate that pigments are precipitating, a desirable property since pigments can be an undesirable impurity in biodiesel.

B. Biodiesel Production

The algal oil collected in the Free Fatty Extraction and Solvent Recycle as outlined in II.A(5) may be converted to biodiesel by esterification, as set forth in U.S. Application No. 61/551,049. This is done by the addition of a strong acid catalyst and an alcohol to the oil. With the addition of heat, the alcohol and catalyst will work to convert the free fatty acids to alkyl esters, also known as biodiesel. Generally this may be done using Sulfuric acid and Methanol, resulting in fatty acid methyl esters or F.A.M.E.s. Once the FAMEs are generated via the esterification reaction, they may be extracted from the reaction mixture using an organic solvent, such as Hexane, and further purified to useable biodiesel. In addition to this method of conversion there are a number of methods that can also be used.

In some embodiments, the steps outlined above may be further simplified and/or combined. For example, in some embodiments, the algal cells may be lysed by any suitable method, including, but not limited to acid hydrolysis. Other methods may include mechanical lysing, such as smashing, shearing, crushing, and grinding; sonication, freezing and thawing, heating, the addition of enzymes or chemically lysing agents. After an initial lysing of the algal cells, the pH is raised as described above in base hydrolysis to form soap from free fatty acids. The resulting aqueous phase which include the soaps in solution is removed, and then a precipitate containing the free fatty acids is formed by lowering the pH as described above in precipitate formation. The lipids may then be extracted by a suitable method, such as those described above.

The following examples are illustrative only and are not intended to limit the disclosure in any way.

### Example II.B.1

Fatty Acid Esterification to Biodiesel. To the residue of Example II.A.5, 1 mL of a 5% (v/v) solution of Sulfuric acid in Methanol was added. This test tube was sealed using a PTFE lined screw cap and the test tube was heated to 90° C. for 30 minutes in a Hach DRB-200 heat block. After 30 minutes the test tube was allowed to cool. Upon cooling 5 mL of Hexane was added to the reaction mixture and the test tube was re-sealed and heated again for 15 minutes at 90° C. FAMEs were extracted into the Hexane phase, which were collected and analyzed for biodiesel content using gas chromatography, or another analytical technique or instrument.

### Example II.B.2

Production Efficiency of Water-Based Lipid Extraction. To test efficiency and the efficacy of heating, the outputs of biodiesel produced according to the methods described herein were tested and compared with a control. Samples were prepared according to the processes described above in Examples II.A.1-5 and II.B.1, with the exception of heat not being added during the various process steps.

The findings are summarized in the data table set forth below.

| | | Standard | |
|---|---|---|---|
| | mg FAME | Deviation (mg) | % of Maximum |
| FAMEs from in-situ TE: | 11.12 | 0.26 | 100% |
| Total FAME Collected: | 10.90 | 0.35 | 98.0% |
| FAME in Hexane Phase: | 6.60 | 0.85 | 59.3% |
| FAME in precipitate: | 1.89 | 0.59 | 17.0% |
| FAME in water phase: | 0.13 | 0.00 | 1.1% |
| FAME in residual | 2.29 | 0.08 | 20.6% |
| biomass: | | | |

“FAME(s)” is the contraction for fatty acid methyl ester(s) also known as biodiesel. FAMEs were quantified using gas chromatography. An Agilent 7890-A GC system equipped with a FID detector was used for this purpose.

“In-Situ TE” refers to a method of transesterification (in-situ transesterification) by which dried algal biomass is directly contacted and subjected to, in this case, Sulfuric acid, Methanol, and heat. This process simultaneously extracts and converts lipids present in the algal biomass to FAMEs or biodiesel. In-situ Transesterification is the method favored, throughout the literature, to measure the biodiesel potential for various types of biomass. This method is considered the control and is assumed to completely convert all present lipids in the algal biomass to FAMEs. Each intermediate collected throughout the process was subjected to this method of FAME production to convert lipids present and quantified by gas chromatography as previously stated.

“Total FAME collected” refers to the sum of FAMEs measured from each intermediate step throughout the process described in this disclosure. This sum is based on averages of three samples, from within the same batch of algal biomass.

“FAME in Hexane Phase” refers to the quantity of FAME collected in the residue remaining after the organic solvent was vaporized.

“FAME in precipitate” refers to the quantity of transesterifiable/esterifiable lipids remaining in the precipitated solid phase, formed in the base neutralization step, after being extracted using the organic solvent and heat.

“FAME in water phase” refers to the quantity of transesterifiable/esterifiable lipids remaining in the aqueous phase after removing the precipitated solid phase.

“FAME in residual biomass” refers to the quantity of transesterifiable/esterifiable lipids remaining in the residual biomass after both hydrolysis steps.

C. Solvent Production

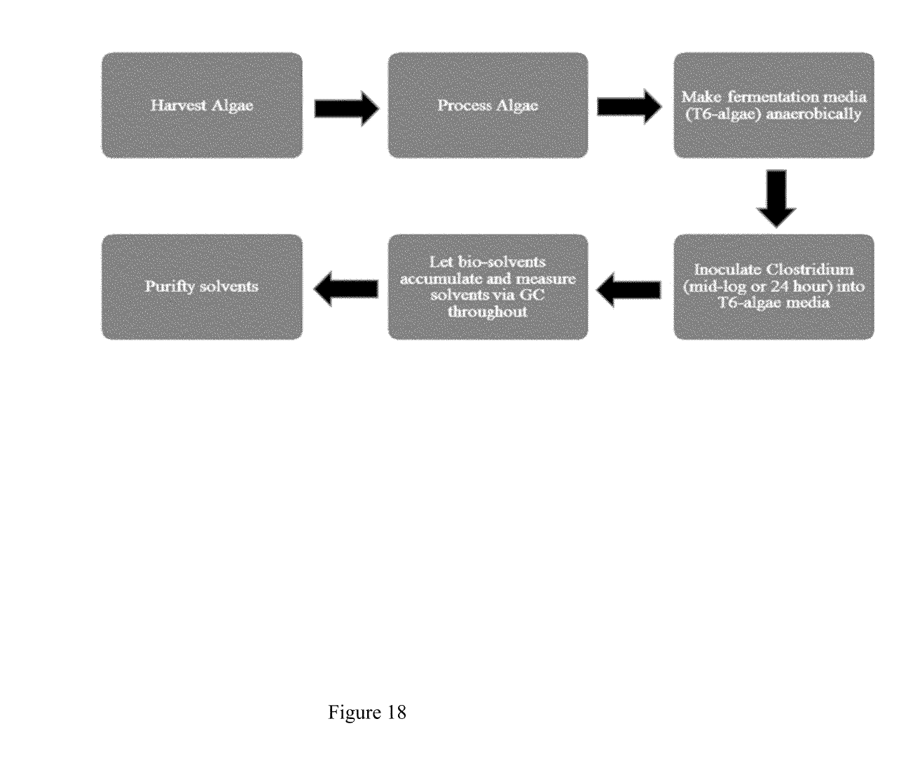

The present disclosure also covers methods, compositions, reagents, and kits for making acetone, butanol, and ethanol (ABE) from algal biomass, some of which are described in U.S. Application No. 61/552,317, filed Oct. 27, 2011, the entirety of which is incorporated by reference in its entirety. A flow diagram of at least one embodiment is illustrated in FIG. 18.

As described above in II.B(1) and II.B(2), after the cells have been lysed, the biomass may be separated from the aqueous solution according to the process described in II.B(3). Once the separation is complete, the water phase is kept separate and the remaining biomass may be optionally washed with water to help remove any residual soap molecules. This wash water may also be collected along with the original liquid phase. Once the biomass is washed it may be taken for solvent production.

The resulting biomass, containing sugars, may then be taken through the exemplary ABE production process described below, or some other suitable ABE production method.

The various steps to the process, according to some embodiments, are described in more detail below. The methods described herein may be accomplished in batch processes or continuous processes.

(1) ABE Production

a. Bacterial Producers

Any suitable bacteria or microorganism capable of metabolizing algal biomass into solvents may be used. At least one Clostridium species or group of species may be used to ferment the algal biomass into ABE. For example, suitable Clostridium species may include, Clostridium saccharoperbutylacetonium, Clostridium acetobutylicum, Clostridium beijerinckii, or any suitable Clostridium bacteria isolated from the environment.

b. Fermentation

ABE fermentation is typically characterized by two distinct phases of metabolism, acidogenesis and solventogenesis. Acidogenesis occurs during log phase of growth, whereas solventogenesis occurs late log phase to early stationary phase of growth. The primary acids produced during acidogenesis are acetic and butyric acid. Clostridia re-assimilate the acids produced during acidogenesis and produce acetone, butanol, and ethanol as metabolic byproducts. The pH-acid effect from acidogenesis plays a key role in the onset of solventogenesis. See, Li et al., Performance of batch, fed-batch, and continuous A-B-E fermentation with pH-control, 102 Bioresource Technology. 4241-4250 (2011).

Any suitable culture medium may be used. Culture medium is used to support the growth of microorganisms, and can be modified to support microbial growth or derive production of certain bio-products. Medium recipes contain vitamins, minerals, buffering agents, nitrogen sources, and carbon sources necessary for bacterial growth. The carbohydrates within algal cells are the carbon source used to drive ABE production throughout the claims. For example, the following culture medium, referred to as T-6, may be used.

### T-6 Medium (Approximate Formula Per Liter)

| Component | Amount | |
|---|---|---|
| Tryptone | 6.0 | g |
| Yeast extract | 2.0 | g |
| KH2PO4 | 0.5 | g |
| MgSO4•7H20 | 0.3 | g |
| FeSO4 7H20 | 10 | mg |
| Ammonium acetate (38.9 mM) | 3.0 | g |
| Cysteine hydrochloride | 0.5 | g |
| Glucose or Algae or other substrate | 5.0-15.0% | (w/v) |

Adjust pH to 6.5 with NaOH

The medium may be formulated to contain about 1 to about 20% processed algae by weight per liter of medium, such as about 4 to about 15%, 5 to about 8%, or 6%.

The other components of the T-6 medium may be varied and adjusted based upon desired growth parameters and/or culturing conditions. In addition, other suitable mediums may include RCM media and TYA media, both of which have been shown to provide suitable nutrients for ABE fermentation with algae as substrate.

The medium may be supplemented with enzymes and/or sugars to help initate primary growth. Suitable enzymes include cellulases and xylanases in amounts ranging from about 10 to about 250 units of enzyme. Suitable sugars include glucose, starch, arabinose, galactose, and xylose in amounts ranging from about 0.1% to about 1.0%.

Once T-6 media constituents are mixed to homogeneity, the media may be neutralized to a pH of about 7, such as about 6.5. The medium may then be modified by any suitable technique to create an anaerobic environment. Suitable techniques for creating such an environment include bubbling the medium with O₂-free N₂ gas for a suitable period of time.

Prior to or after the creation of the anaerobic environment, the medium may be optionally sterilized.

The medium may be inoculated with at least one Clostridium species. The concentration of bacteria may be varied, depending on the culture vessel and scale of the fermentation. Prior to or after inoculation, the bacterium may be heat shocked to a temperature of about 70° C. for a suitable period of time to germinate the spores. The bacterium may also be incubated in a growth medium at optimal temperature prior to inoculation to allow the spores to become vegetative prior to transferring to the growth medium. After inoculation, the fermentation vessel head space, if any, may be flushed with N₂ gas to ensure optimal anaerobic growth conditions.

The culture may be incubated at about 35° C. throughout. Typically, 48 hours is needed for T-6 glucose cultures containing spores of Clostridium saccharoperbutylacetonium to reach mid-log phase, though fermentation times may vary depending on the vessel size, inoculation concentration, and temperature. T-6 algae media fermentations may be conducted for about 96 hours to reach optimal ABE production. T-6 glucose fermentations may be used as the positive control, whereas T-6 media without a carbon source may be used as the negative control throughout.

(2) ABE Purification

Any suitable purification method may be employed. In some embodiments, distillation may be used for purifying the various fermentation products. Distillation is used widely for alcoholic beverages, as well as for other types of fermented solutions, particularly acetone, butanol, and ethanol. When distillation is employed, purification is accomplished based on different boiling points from one compound to another. By heating a mixture to a temperature just above each solvents boiling point, the desired compound evaporates and then condenses independently to acquire purified solvents.

In some embodiments, each of the fermentation products may be purified; however, in other embodiments, only a select product or group of products may be purified. In particular, because the yield for acetone and butanol are higher than that of ethanol, some purification processes only purify acetone and butanol, while other fermentation products are flared off or otherwise discarded.

Other suitable purification methods may be employed, such as absorption, membrane pertraction, extraction, and gas stripping. See, e.g., Kaminski et al., Biobutanol—Production and Purification Methods, Ecological Chemistry and Engineering S., Vol. 18, No: 1 (2011).

The following examples are illustrative only and are not intended to limit the disclosure in any way.

### Example II.C.1

Biomass Processing. To a glass test tube 100 mg of lyophilized algal biomass was added. One mL of a 1 Molar Sulfuric acid solution is added to the test tube and the test tube was then sealed using a PTFE lined screw cap and gently mixed to create a homogenous slurry. This slurry was then placed in a Hach DRB-200 heat block pre-heated to 90° C. This slurry is allowed to digest for 30 minutes with mixing at the 15 minute mark.

Once the first 30 minute digestion period was completed, the test tube was removed from the heat source and 0.75 mL of a 5 Molar Sodium Hydroxide solution was added to the test tube. The test tube was immediately resealed and returned to the heat source for 30 minutes. Mixing at 15 minutes was again provided.

Once the base hydrolysis above was completed, the test tube was removed from the heat source and allowed to cool in a cold water bath. Once cooled the test slurry was centrifuged using a Fisher Scientific Centrific Model 228 centrifuge. The upper aqueous phase was removed and collected in a separate test tube. To the remaining biomass 1 mL of deionized water as added and vigorously mixed. The slurry was re-centrifuged, and the liquid phase collected and added to the previously collected liquid phase. The liquid phase was then removed from the process and processed biomass was taken for further processing.

### Example II.C.2

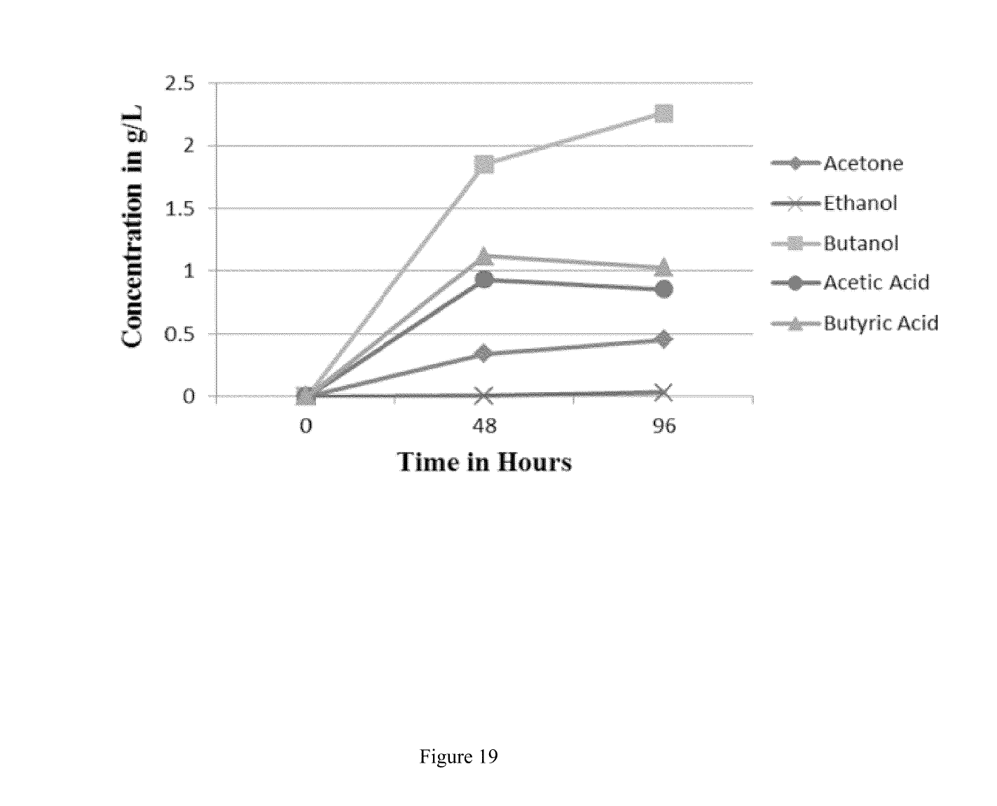

ABE production using processed biomass and no supplementation of enzymes or sugar. 10% algal biomass was processed according the parameters described in Example II.C.1. The T-6 media constituents were mixed to homogeneity, and the media neutralized to pH 6.5, and the media was then dispensed into serum vials. These vials were then bubbled with O₂ free N₂ gas for 10 minutes to remove any O₂ (thus generating an anaerobic environment). Once this was performed, the vials were sealed, crimped, and sterilized. After sterilization, 1 ml of a concentrated spore suspension containing Clostridium saccharoperbutylacetonium was transferred to T-6 glucose media anaerobically. After inoculation, the growth media containing spores was heat shocked at 70° C. for 10 minutes to germinate spores and incubated at optimal temperature. This step allowed the spores to become vegetative prior to transferring into T-6 algae media. After the T-6 glucose culture reached mid-log phase, a 10% inoculum of mid-log phase cells was transferred into T-6 algae media (containing 10% processed algae) anaerobically. After fermentation media was inoculated, the head space was flushed with O₂ free N₂ gas for 5 minutes to ensure optimal growth conditions and O₂ removal. The culture was then incubated at 35° C. throughout for 48 hours to reach mid-log phase. The fermentation was conducted for 96 hours to reach optimal ABE production. The mean yield results of two replicates of are illustrated in FIG. 19.

### Example II.C.3

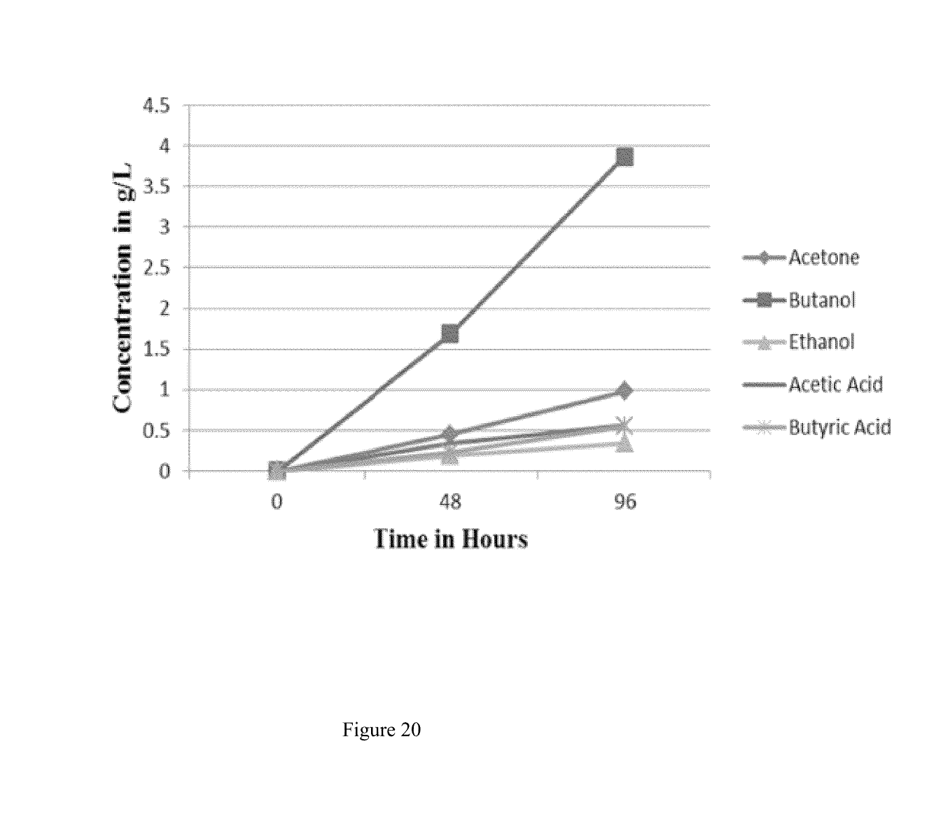

ABE production using processed biomass and enzymes. 10% algal biomass was processed according the parameters described in Example II.C.1. The process biomass was fermented as described in Example II.C.2 with the supplementation of 250 units of xylanase and 100 units of cellulose added to the fermentation. The yield results are illustrated in FIG. 20.

### Example II.C.4

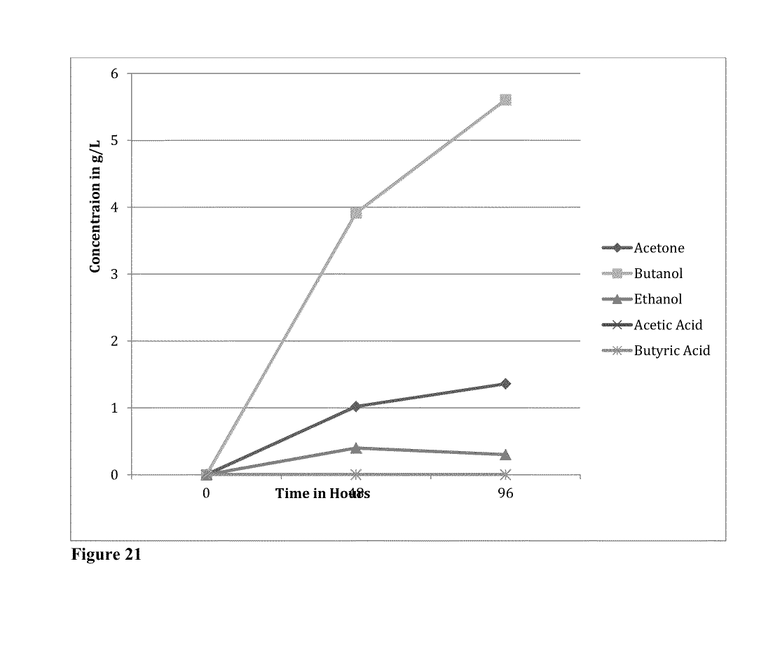

ABE production using processed biomass and sugar. The same process as described in Example II.C.2 was repeated, this time supplementing only with 1% dextrose. The yield results are illustrated in FIG. 21.

### Example II.C.5

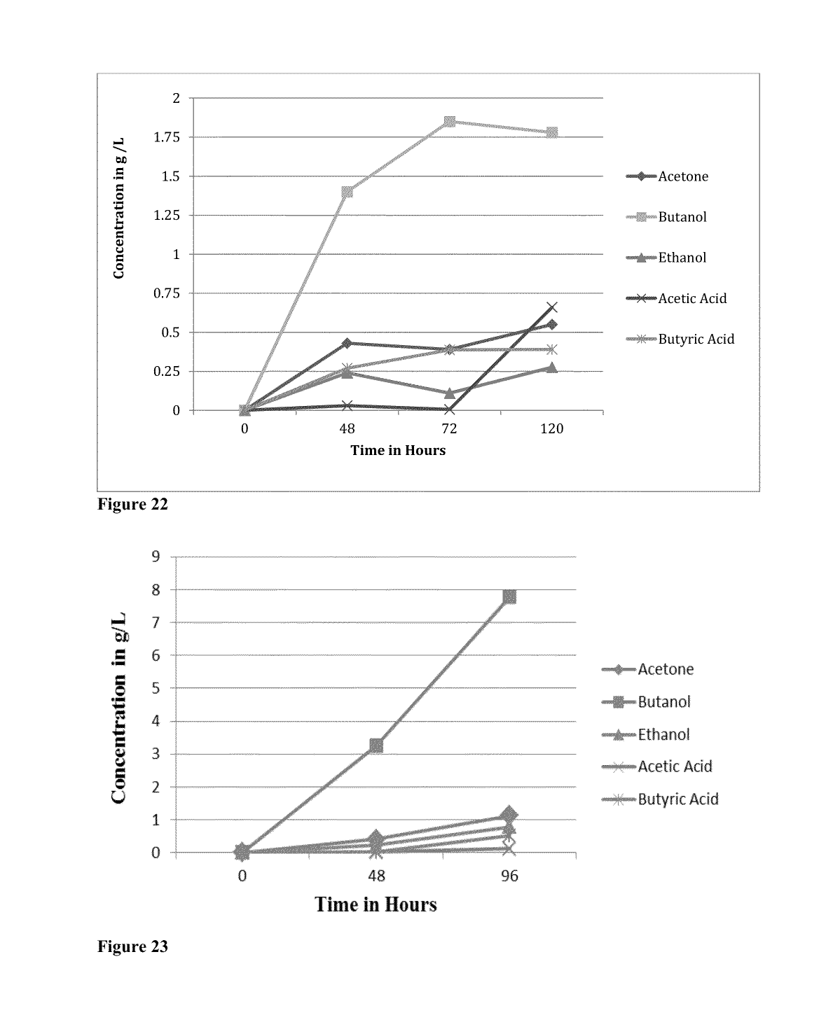

ABE Production using pretreated algae and enzymes. Dried algae was crushed using a blender and then pretreated with 250 mM sulfuric acid for 30 min at 120° C. Acid and solvent production from Clostridium saccharoperbutylacetonium using 10% algae supplemented with xylanase and cellulase enzymes as described in Example II.C.2 was undertaken. The yield results are illustrated in FIG. 22.

### Example II.C.6

ABE Production using pretreated algae and enzymes. Dried algae was crushed using a mortar and pestle and then pretreated with 250 mM sulfuric acid for 30 min at 120° C. Acid and solvent production from Clostridium saccharoperbutylacetonium using 10% algae supplemented with xylanase and cellulase enzymes as described in Example II.C.2 was undertaken. The yield results are illustrated in FIG. 23.

### Example II.C.7

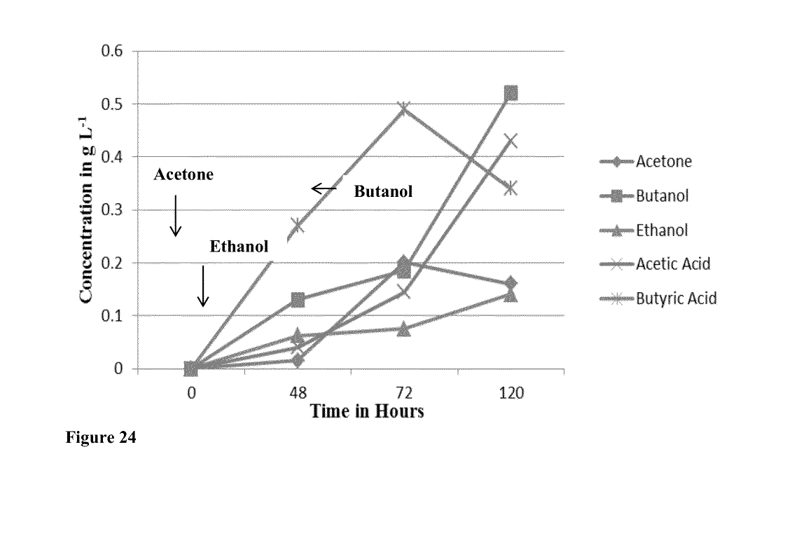

ABE production using non-pretreated whole cell algae. Dried algae was used in T-6 media without any chemical or mechanical modifications to the algae cells. The algae was fermented according to the fermentation conditions outlined in Example II.C.2, except that dried, unprocessed algae was used and a 5% inoculum was used for a 24 hour culture in RCM media. The yield results are illustrated in FIG. 24.

### Example II.C.8

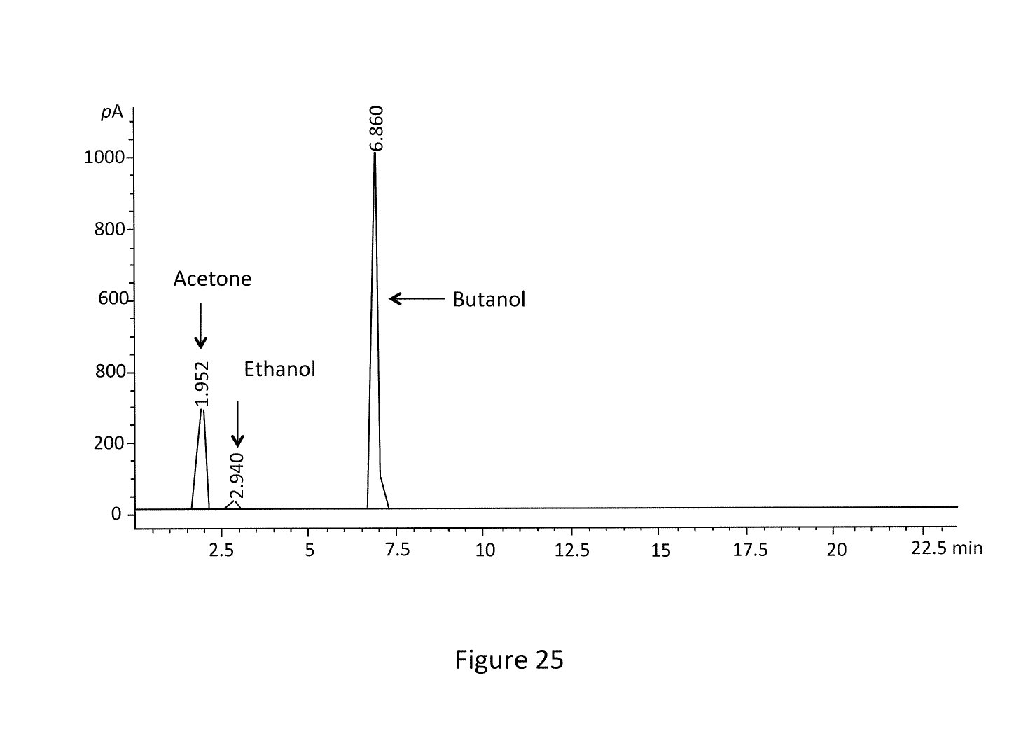

Gas Chromatography (GC). A GC chromatogram, used to measure or quantify ABE, using clarified culture supernatant the method described in Example II.C.4 is shown in FIG. 25. The protocol for measuring ABE via GC analysis is as follows:
Instrument: Agilent Technologies 7890A GC system.
Column specs: Restek Stabiwax-DA, 30 m, 0.32 mmID, 0.25 um df column.
Inlet: initial 30 C for 1 min; ramp 5 C/min up to 100 C; ramp 10 C/min up to 250 C.
Column: flow 4 ml/min; pressure 15.024 psi, Avg velocity 53.893 cm/sec; holdup time 0.92777 min.
Oven: initial 30 C for 1 min; ramp 5 C/min up to 100 C (no hold time); ramp 20 C/min up to 225 C (no hold time); ramp 120 C/min up to 250 C and hold for 2 min.
FID: Heater at 250 C; H2 flow at 30 ml/min; Air flow at 400 ml/min; makeup flow (He) at 25 ml/min.
Miscellaneous: 1 μl injection volume, and Helium as carrier gas.

D. Bioplastic Production

The present disclosure also covers methods, compositions, reagents, and kits for making bioplastics from algal biomass, some of which are described in U.S. Provisional Application No. 61/657,649, filed Jun. 8, 2012, the entirety of which is incorporated by reference in its entirety.

(1) Feedstocks

As a feedstock, any suitable algae may be used. In embodiments, algae that produce high concentrations of polysaccharides may be preferred. In many embodiments, algae produced in wastewater may be used. The algae may be lyophilized, dried, in a slurry, or in a paste (with for example 10-15% solid content).

Any suitable algae harvesting method may be used alone or in combination with one another. For example, the algae may be harvested using a rotating bioreactor, as described in U.S. patent application Ser. No. 13/040,364 (herein incorporated by reference in its entirety). In addition to or independent from, the algae may be harvested using inorganic or organic coagulants/flocculants as described in U.S. Provisional Patent Application 61/552,604 (herein incorporated by reference in its entirety).

When organic coagulants/flocculants are used, the feedstock will include both the algae and the organic coagulant/flocculant.

(2) Flocculation

In embodiments, organic coagulants and flocculants, as described in Section MB above, may be employed to effectively harvest algae without negatively affecting the various bio-products that may be later derived from algae.

After identification and/or harvesting of a feedstock source or sources, the algae may be formed into a slurry, for example, by adding water, adding dried or lyophilized algae, or by partially drying, so that it has a solid content of about 1-40%, such as about 4-25%, about 5-15%, about 7-12%, or about 10%.

(3) Algal Biomass Pre-Processing

In some embodiments, the feedstock may optionally be pre-processed using WLEP as described above in Section II.A. As such, the cells in the feedstock are lysed, followed by biomass and aqueous phase separation and precipitate formation as described in Section II.A.

In such embodiments, after the biomass is removed, the pH of the collected liquid may be neutralized/reduced to form a precipitate. This may be accomplished by the addition of an acid to the solution, such as at least one strong acid or mineral acid, for example, sulfuric, hydrochloric, phosphoric, or nitric acid. Addition of a suitable acid is performed until a green precipitate is formed. The green precipitate may contain, or may be, the Porphyrin heads as they are converted from their salt forms. It may also contain proteins and other cellular components that are coming out of solution.

The pH may be reduced to a pH of about 7 or less, such as about 4-6.9. This lower pH also converts the soap in the liquid to free fatty acids. As the precipitate forms the fatty acids associate with the solid phase and come out of solution. Once the precipitate has formed, the solid and liquid phases may be separated. Any suitable separation method may be employed, such as centrifugation, gravity sedimentation, filtration, or any other form of solid/liquid separation. The liquid phase may be taken for further processing into bioplastics production. The collected solid phase may be removed and further processed into other useful products, such as biodiesel as described above.

(4) Bioplastic Production

(a) Bacteria

Any suitable bacterial strain capable of producing bioplastics may be used. For example, the Escherichia coli strain described in U.S. patent application Ser. No. 12/907,572, filed Dec. 19, 2010, the entirety of which is herein incorporated by reference.

(b) Growth Medium

The liquid/aqueous phase may be used directly as a medium for growth of bacteria capable of producing bioplastics or any other bioproducts. The liquid phase may be optionally augmented with other growth mediums and/or components, such as liquids, nutrients, minerals, and growth factors. The growth medium may contain at least 0.1% glycerol, such as at least 0.5% glycerol, or from 0.1 to about 20% glycerol, or from about 0.5 to about 15%. In addition to glycerol the liquid/aqueous phase may also contain other (undefined) simple sugars that the bioplastics-producing microbe can use as a carbon source. Furthermore, the liquid/aqueous medium is at an optimum salt/ion concentration which provides the ideal buffering capacity for the bacteria to grow and produce PHB. The liquid media also does not inhibit the effect of antibiotics or the inducer Isopropyl β-D-1-thiogalactopyranoside (IPTG), which are required for the maintenance of the pBHR68 plasmid and the start of PHB gene expression respectively. In some embodiments, the growth medium may be used alone or in combination with other growth mediums for fermenting any bacterial strain that requires a sugar source for growth.

(c) Growth of Bacteria

The bacteria may be grown or fermented in the growth medium at a suitable temperature for a suitable period of time to maximize production of bioplastics. Fermentation may be undertaken in small or large fermenters in either a batch or continuous setup. Typically, the bacteria are grown at about 37° C. for a period of about 1 to 4 days, such as about 48 hours.

(d) Purification

After fermentation, the bioplastics may be purified from the medium depending on the bacteria strain used. In some embodiments, the bacteria may be separated from the growth medium (which may be optionally or partially recycled) by a suitable separation method, such as filtration, centrifugation, etc.

Any suitable purification technique may be used. The PHB may be directly quantified using the NMR/GC method outlined in the Examples below. In such a method, bacterial cells may be subjected to bleach and chloroform. The bleach lyses open the cells, liberating the PHB into the chloroform phase. In embodiments using PHB secreting bacteria, the bacterial culture was treated with CaCl₂ to separate the secreted PHB from the non-secreted PHB.

(e) Examples

The following examples are illustrative only and are not intended to limit the disclosure in any way.

E. coli strain harboring the pBHR68 plasmid was cultured in culture medium derived from the algal strains associated with or without flocculants as follows:

| Sample | Algae Strain | Flocculent |
|---|---|---|
| 1 | Scenedesmus obliquus | Aluminum Sulfate |
| 2 | Scenedesmus obliquus | Modified potato starch |
| 3 | Scenedesmus obliquus | None |
| 4 | Logan Lagoons Algae | Modified corn starch |
| 5 (control) | None | None |
| 6 | Logan Lagoons Algae | Centrifuged |
| 7 | Logan Lagoons Algae | Aluminum Sulfate |
| 8 | Logan Lagoons Algae | Modified potato starch |

Ten sample culture mediums were derived by performing acid hydrolysis, base hydrolysis, biomass and aqueous phase separation, and pellet formation as described above to produce a liquid phase from the above feedstock materials. Once products were received, each sample had 100 mL centrifuged at 3500 rpm for 25 min. The supernatant was placed in a beaker and pH was adjusted to approximately pH 7 with NaOH. It should be noted that all samples had an initial pH of less than 3 before neutralization. These neutralized samples were then divided into separate flasks (100 ml of each sample in each flask). Each flask was autoclaved at 121° C. for 25 min.

The control flask consisted of 20 mL solution of 10 g YE+75 g glucose per L)+10 mL 10×M9+0.02 mL MgSO₄+70 mL H₂O.

To each sample flask was added 1004, Amp50, 1004, IPTG, 1 mL pBHR68 (non-secreting). The flasks were placed at 37° C. on a shaker table and bacterial growth (colony forming units CFU/mL) was measured at 0, 4, 8, 12, 24, and 48 hrs. After 48 hours samples were centrifuged at 3500 rpm for 25 min. The resulting pellet was then freeze dried for 48 hours. Freeze dried samples were then processed for NMR analysis. An NMR-GC correlation was used to determine the PHB concentration in each sample. See E. Linton, A. Rahman, S. Viamajala, R. C. Sims, C. D. Miller, Polyhydroxyalkanoate quantification in organic wastes and pure cultures using a single-step extraction and 1H NMR analysis, Water Science and Technology, Accepted Manuscript (2012).

The results of these samples are summarized below:

Medium for Growth
After neutralization of the aqueous phase from WLEP, it can be used as a suitable medium for bacterial growth.
While the dominate carbon source is expected to be glycerol, there could be other simple sugars in the media that aid in growth.
There are micronutrients (such as salts) in the aqueous phase that provide a suitable medium for bacterial growth.

Bacterial Growth and Viability
Bacterial growth was seen for all samples.
Bacterial growth (CFU/mL) was calculated for all samples. Samples grown in the aqueous phase from single strain algae (Scenedesmus obliquus) had higher CFU/mL on average than samples grown in Lagoon algae aqueous phase.

Bioplastic Production
Bacterial growth was seen in alum samples. However, no PHA production seen in these samples. This could mean that PHA being produced is below the detection limit of the NMR.
PHB was seen in single strain Scenedesmus obliquus flocculated with potato starch and processed with WLEP. From this it can be assumed that all other algae strains will act similarly.
PHB was seen in single strain Scenedesmus obliquus with traditional centrifugation and processed with WLEP
Bioplastic was seen in Logan Lagoon algae flocculated with corn starch and processed with WLEP (partially addresses the objectives outlined in overall Lagoon/combined patent).
Yields of bioplastic from processed single strain and mixed algae were similar (without replicates), however these yields were less than that seen in the control.

Laboratory Grade Glycerol
When compared to LB control, bioplastics-producing bacteria growing in M9-glycerol did not reach the same OD.
It was shown with NMR spectra that PHB can be produced using glycerol as the sole carbon source.

Determination of Glycerol Concentration in Aqueous Phase
From using a commercial kit (Biovision free glycerol assay kit), the aqueous phase was found to have 0.05 g/L concentration of glycerol.
In addition, there could be other simple sugars in the aqueous phase that still need to be analyzed. These simple sugars could have aided in the growth of bacteria.

The results are summarized in the following table (PHB yields were calculated using NMR/GC correlation):

| Sample/ | | | |
|---|---|---|---|
| Flask | | PHB peaks | Concentration |
| number | Description | present? | mg/mL |
| 1 | Alum only, Algae source: | No | |
| | Scenedesmus Obliquus | | |
| 2 | Potato starch only, | Yes | 0.086 ± 0.032 |
| | Algae source: | | |
| | Scenedesmus Obliquus | | |
| 3 | centrifuged, Algae source: | Yes | 0.089 ± 0.027 |
| | Scenedesmus Obliquus | | |
| 4 | Corn, Algae source: Logan | Yes | 0.084 ± 0.014 |
| | Lagoons | | |
| 5 | enhanced M9 media | Yes | 0.38 ± 0.05 |
| 6 | centrifuged, Algae source: | Yes | 0.044 ± 0.014 |
| | Logan Lagoons | | |
| 7 | Alum only, Algae source: | No | |
| | Logan Lagoons | | |
| 8 | Potato starch only, | Yes | 0.070 ± 0.035 |
| | Algae source: Logan | | |
| | Lagoons | | |

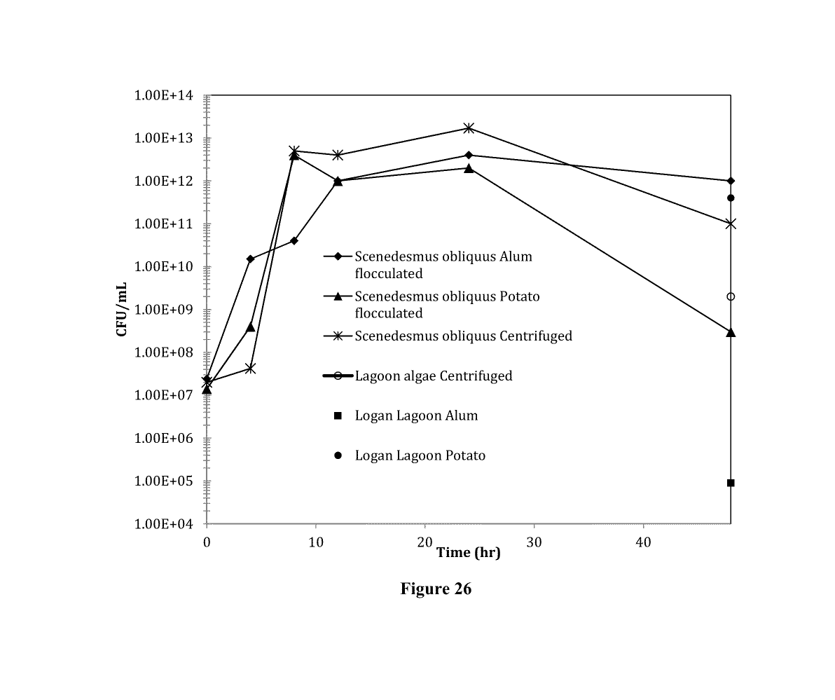

FIG. 26 illustrates CFU/mL for Samples 1-3 and 6-8.

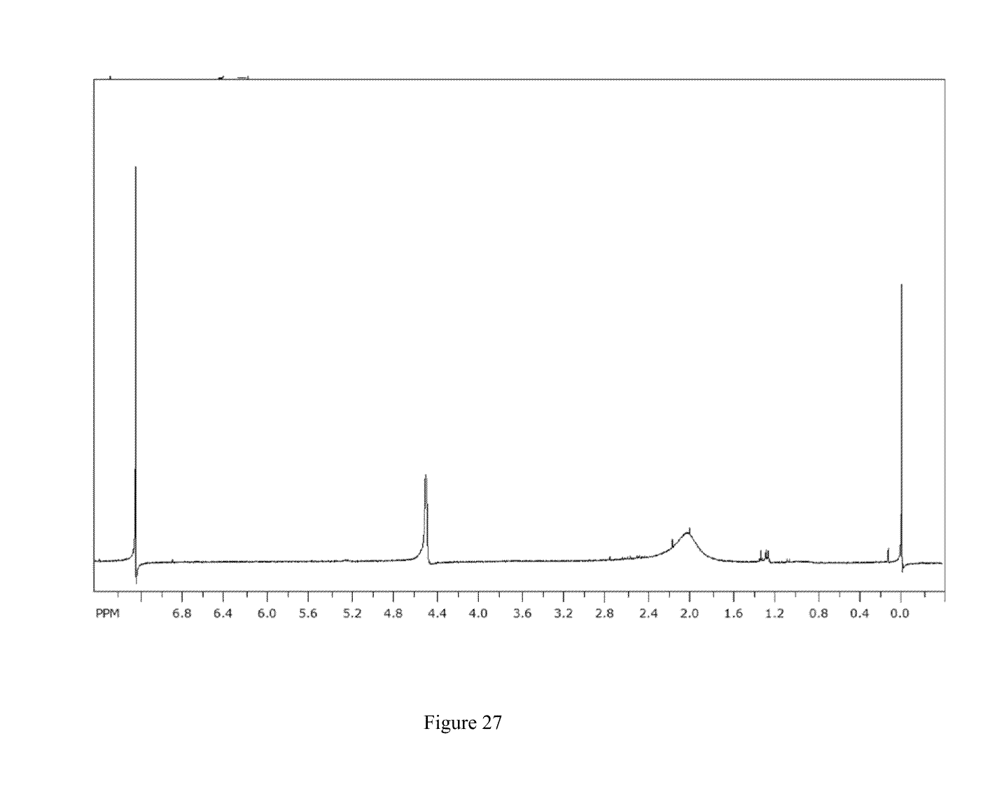

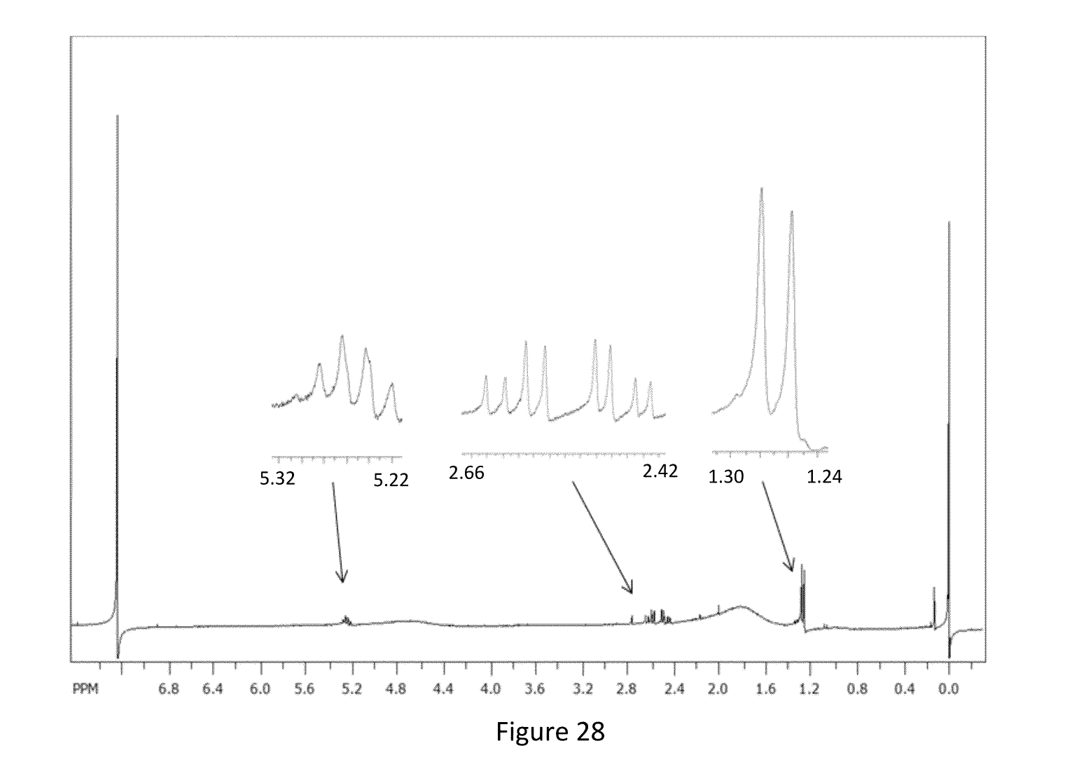

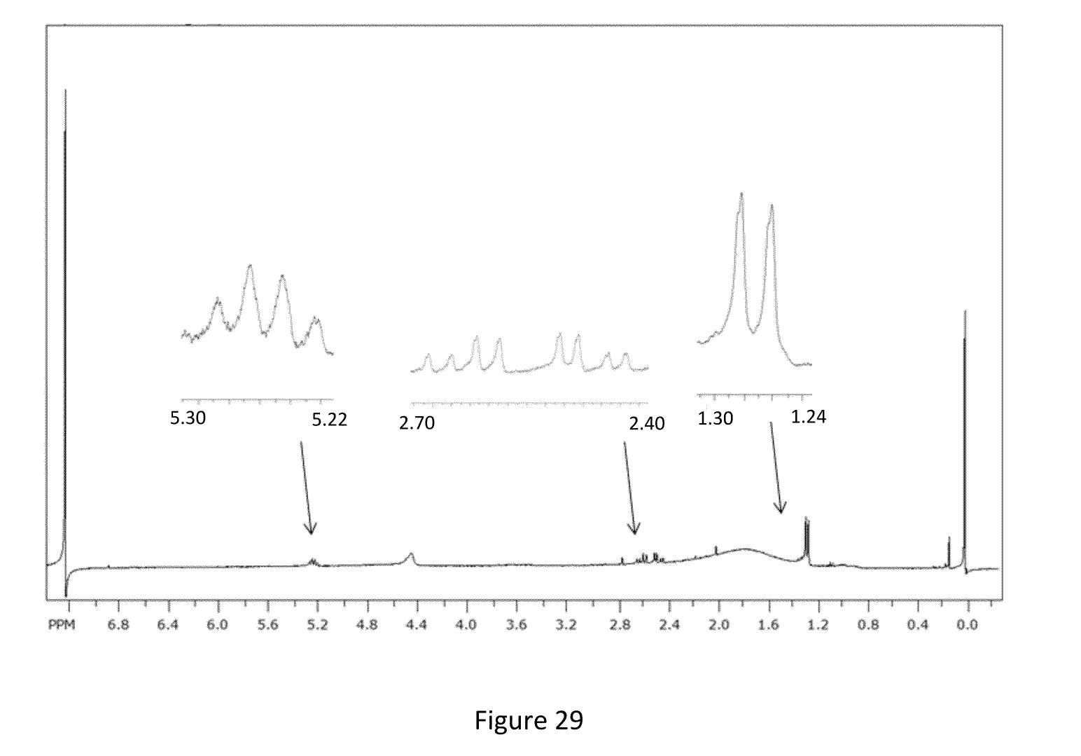

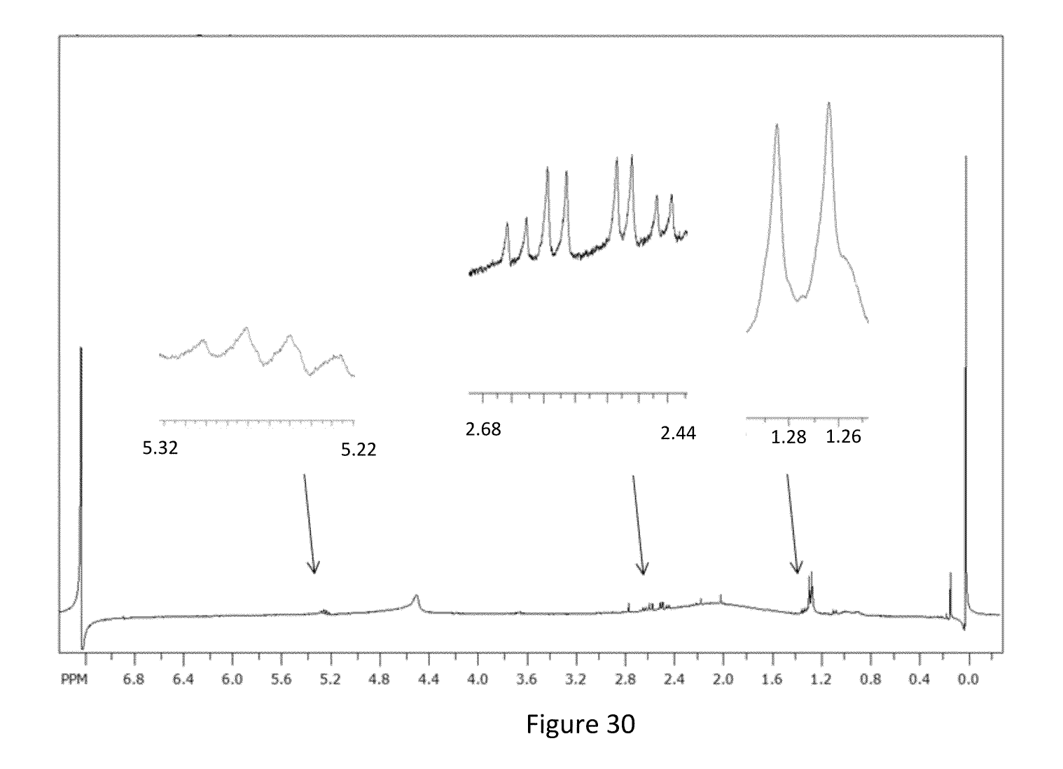

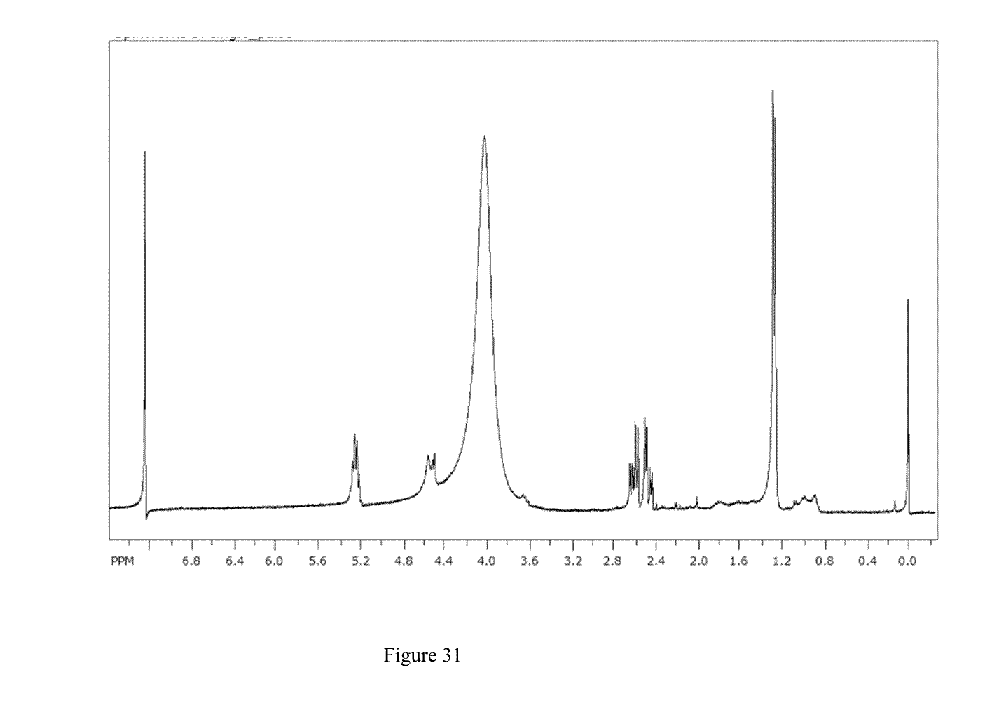

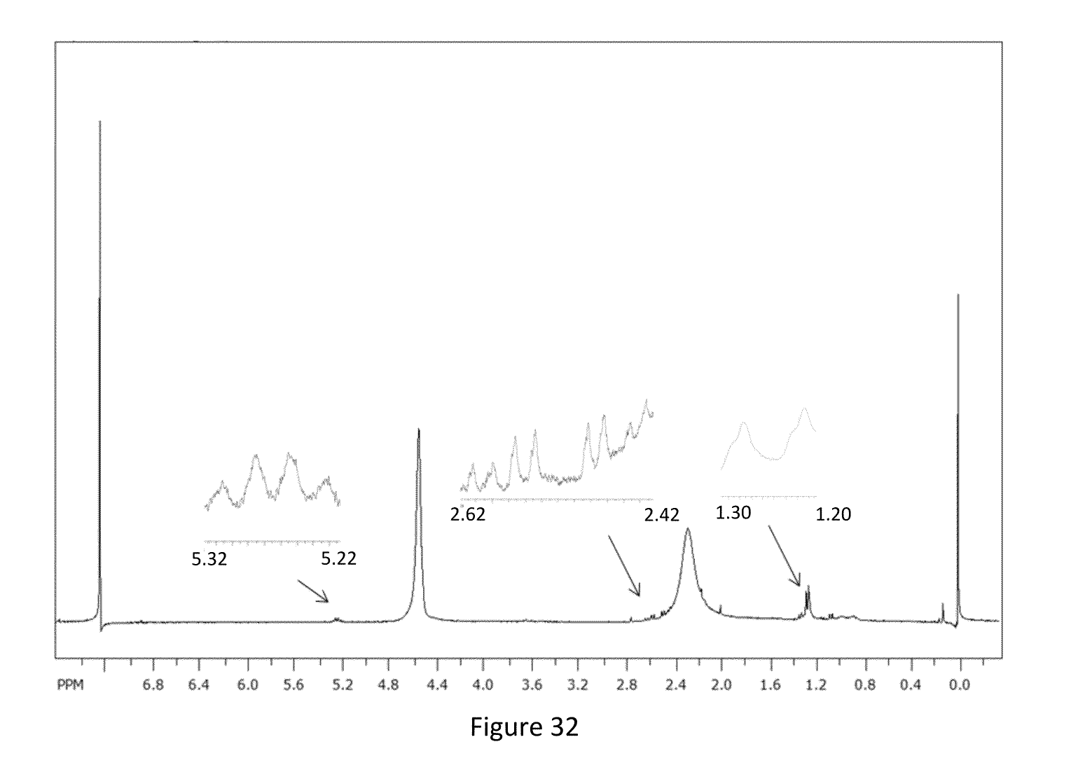

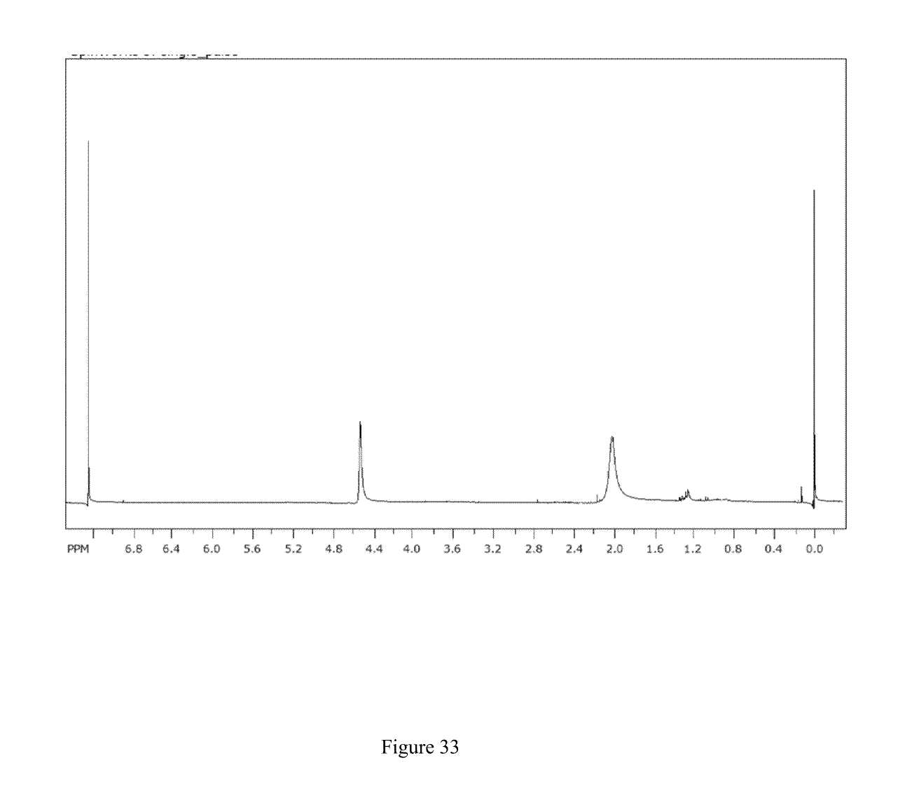

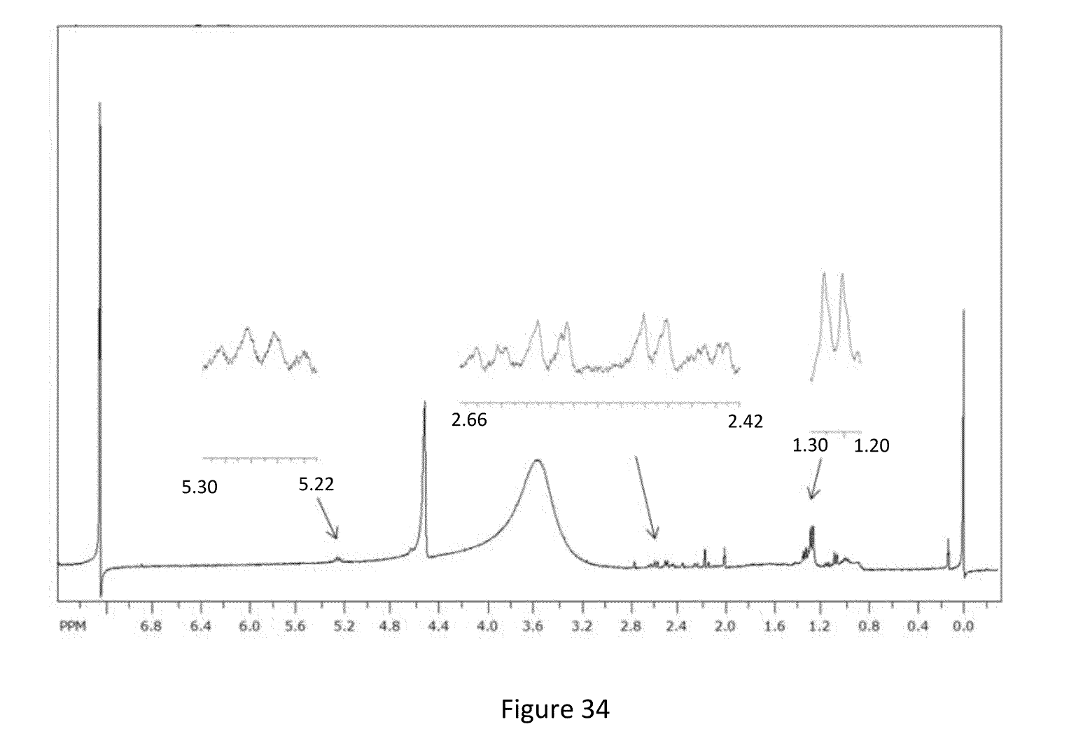

NMRs of Samples 1-8 are respectively illustrated in FIGS. 27-34.

Pure Glycerol Example:

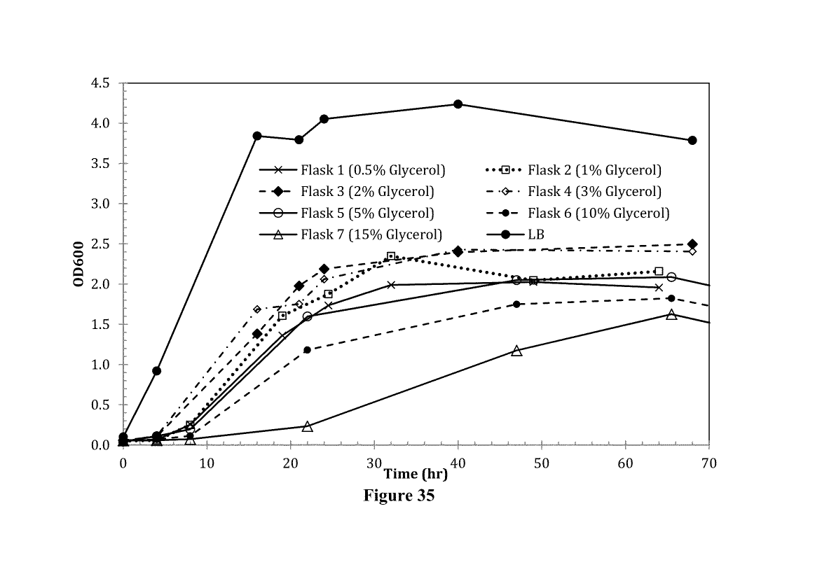

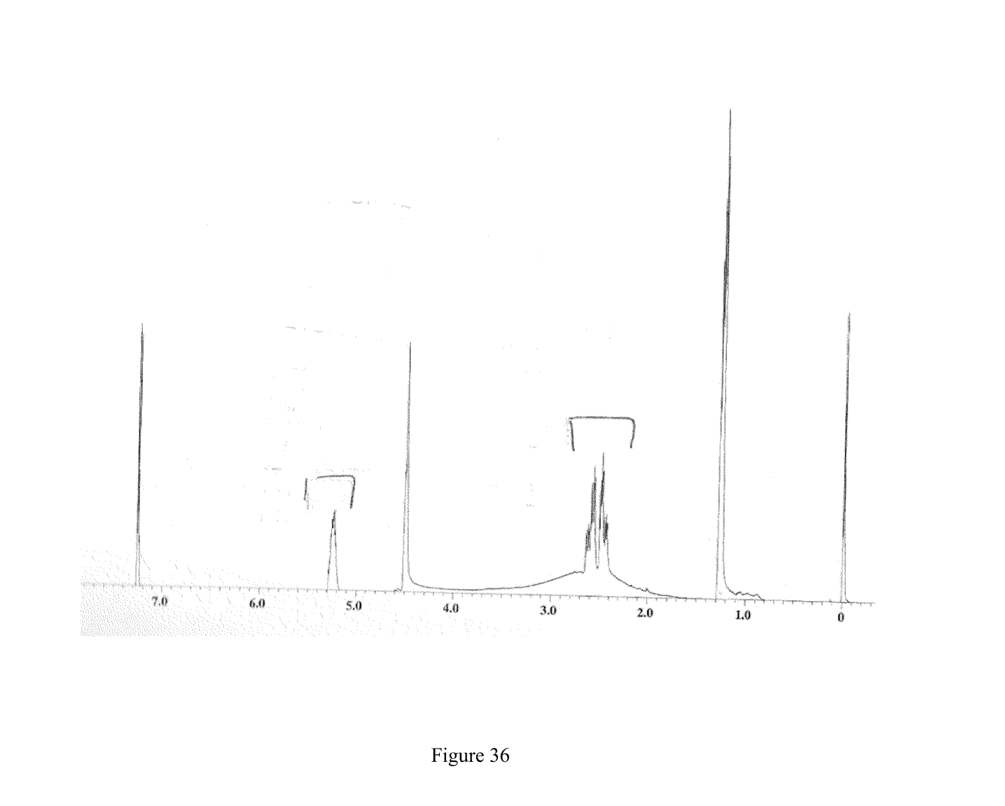

Bacterial strains harboring the plasmids 4MHT in pBHR68+pLG575 were grown in M9-glycerol media. The results are illustrated in FIGS. 35 and 36. From this example it is shown that PHB may be generated from laboratory grade glycerol. This example demonstrates growth of PHB producing strains on different concentrations of glycerol (0.5-15%).

E. Anaerobic Digestion

As illustrated in FIG. 1, a portion of or all of the washed or unwashed biomass resulting from WLEP may be further processed by anaerobic digestion to produce methane gas and/or fertilizer components. In some embodiments, the methane gas may be recycled back into the system to power system components, such as boilers, the rotating bioreactors, etc. Any suitable anaerobic digester may be used. The biomass may be supplemented with algae or other biomass that is not preprocessed or is at any state of WLEP.

III. Overall System

The above disclosure sets for details relating to harvesting (Section I) and processing (Section 2). More specifically, it sets forth details for mechanical harvesting (Section I.A), chemical harvesting (Section I.B), WLEP (Section II.A), biodiesel production (Section II.B), biosolvent production (Section II.C), bioplastics production (Section II.D), and biogas and fertilizer production (Section II.E).

These parts, or modules, may be integrated in any combination. Exemplary systems may include all of the modules but be configured to turn on or off particular modules based on economic drivers and/or processing product needs. Thus, the system may be designed to be flexible and provide optimum outputs based on the needs of the system operator. The modules described herein and the overall system may be implemented in any system needing to manage algal growth. In particular, this system may be employed in water treatment plants and/or “lagoon” water treatment systems.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A system for harvesting algae, comprising:
a mechanical harvesting system, and
a chemical harvesting system.

2. The system of claim 1, wherein the mechanical harvesting system comprises a rotating bioreactor.

3. The system of claim 1, wherein the chemical harvesting system comprises organic coagulants.

4. The system of claim 3, wherein the organic coagulants comprise modified starch.

5. A system for harvesting and processing algae, comprising:
a rotating bioreactor harvester,
a chemical harvesting module,
a biodiesel producing module,
a biosolvent producing module,
a bioplastics producing module, and
a biogas and fertilizer producing module.

6. A method for harvesting and processing algae, the method comprising:
harvesting algae, and
processing algae.

7. The method of claim 6, wherein harvesting algae comprises harvesting algae with a rotating bioreactor.

8. The method of claim 6, wherein harvesting algae comprises harvesting algae with organic chemical coagulants.

9. The method of claim 6, wherein processing algae comprises wet lipid extraction.

10. The method of claim 9, wherein processing algae further comprises producing biodiesel.

11. The method of claim 9, wherein processing algae further comprises producing biosolvents.

12. The method of claim 9, wherein processing algae further comprises producing bioplastics.

13. The method of claim 9, wherein processing algae further comprises producing biogas and/or fertilizer.
